(19) United States
(12) Patent Application Publication
(10) Pub. No.: US 2024/0344006 A1
(43) Pub. Date: Oct. 17, 2024

(54) METHOD OF MANUFACTURE FOR EDIBLE, POROUS CROSS-LINKED HOLLOW FIBERS AND MEMBRANES BY PH INDUCED PHASE SEPARATION AND USES THEREOF

(71) Applicant: MERCK PATENT GMBH, Darmstadt (DE)

(72) Inventors: Kevin T. Dicker, Fox Point, WI (US); Ryan Sylvia, Tewksbury, MA (US); Aletta Schnitzler, Melrose, MA (US); Jaivin Patel, Burlington, MA (US); Luca Cera, Cambridge, MA (US)

(21) Appl. No.: 18/291,524

(22) PCT Filed: Aug. 19, 2022
(86) PCT No.: PCT/EP2022/073261

§ 371 Date: Jan. 23, 2024

Related U.S. Application Data
(60) Provisional application No. 63/234,796, filed on Aug. 19, 2021.

(51) Int. Cl. C12M 1/12; B01D 67/00; B01D 69/02; B01D 69/06; B01D 69/08; B01D 71/08; B01D 71/74

(52) U.S. Cl. CPC C12M 25/02; B01D 67/0011; B01D 67/00165; B01D 67/0083; B01D 67/0086; B01D 67/0088; B01D 67/009; B01D 67/0093; B01D 69/02; B01D 69/06; B01D 69/08; B01D 71/08; B01D 71/74; C12M 25/10; B01D 2323/081; B01D 2323/10; B01D 2323/12; B01D 2323/20; B01D 2323/21811; B01D 2323/21826; B01D 2323/219; B01D 2323/30; B01D 2323/34; B01D 2325/56; B01D 2325/58

(57) ABSTRACT

A method of manufacture of crosslinked, edible, porous hollow fibers and sheet membranes suitable for the manufacture of clean meat products, the hollow fibers and sheet membranes made therefrom and methods of use thereof.

### CROSS REFERENCE TO RELATED APPLICATIONS

This application claims the benefit of priority of U.S. Provisional Patent Application No. 63/234,796, filed Aug. 19, 2021, the entire contents of which are hereby incorporated by reference in its entirety.

### BACKGROUND OF THE INVENTION

Membrane integrity and pore properties are paramount for effective use in membrane-based bioreactors. Membranes need to be self-supporting to allow for the transfer of media and nutrients through the membrane without interfering support structures and to allow for greater surface area for the culturing of adherent cells. Further, for the production of edible food stuffs, membranes need to be made of materials generally recognized as safe (GRAS). Still further, making membranes which are edible, both from a technical aspect (i.e., non-toxic and digestible) and from a practicable, consumer acceptable aspect (i.e., having texture and mouth feel acceptable to consumers) has not been achieved in the art. The production of such membranes, whether flat sheet (for example, nano porous membranes) or fibers (for example, hollow fibers) has been elusive. What is needed are membranes of high integrity for use in membrane-based bioreactors that are suitable for cell culture and are edible.

### SUMMARY OF THE INVENTION

The present inventors have developed a novel and non-obvious method of making membranes (i.e., membrane films and fibers) by, for example, pH induced phase separation or proton induced phase separation that have the requisite structural integrity for use in bioreactors for the production of food stuffs for human and animal consumption. The membranes are made with materials GRAS, are self-supporting (i.e., do not collapse on to themselves and do not easily tear or easily rip when handled or exposed to fluid forces necessitated by culture conditions in a bioreactor) and are edible both from technical and from practicable, consumer acceptable aspects.

The membranes of the present invention, in the broadest embodiment, comprise one or more plant or animal proteins, one or more edible polysaccharides and, optionally, one or more polysaccharide crosslinking agents. The protein(s), polysaccharide(s) and optional crosslinking agent(s) are co-mixed and extruded into a formation bath. The formation bath contains one or more ions (i.e., cations or anions) which result in the crosslinking of the polysaccharides in the membrane. Additionally, in some aspects of the present invention, pH changes in the formation bath result in phase separation induced membrane formation.

The present inventors have learned empirically that crosslinking of the polysaccharides in the membrane is often insufficient to ensure adequate membrane integrity especially under cell culture conditions (see, Exemplification). The present inventors have further invented a process for imparting the membranes with the requisite integrity. After formation of the membranes in the formation bath, the membranes are then exposed to an energy source such as heat or irradiation. While not limited by theory, the present inventors believe that exposure to the energy source results in crosslinking of the polysaccharide and/or proteins in the membrane thereby providing the requisite integrity to the membrane while maintaining qualities needed for consumer acceptance.

Also a consideration with regard to providing membranes for use in foodstuffs, prior art techniques of chemical crosslinking often uses toxic compounds which will need to be avoided for this application. Alternatively, prior art polymer modification techniques may be used for increasing crosslinking sites but may run into regulatory challenges.

In another aspect, the membranes of the present invention may be coated or otherwise modified with one or more agents to, for example, enhance cell attachment and cell growth. The membranes may be coated prior to or after exposure to heat or irradiation.

After formation, exposure to an energy source and optional coating, the membranes may be partially dried and/or stored or subject to further processing (for example, by being cut to size and incorporated into a bioreactor cartridge or capsule).

Accordingly, the invention relates to edible 3D nano and micro porous structures for use in membrane bioreactors (film or fiber-based) for the production of, for example, structured clean meat products. Culture media passes through the membrane to feed the cells on one or both surfaces of the membrane. Prior art hollow fiber membrane bioreactors exist for adherent cells, but trypsin or other chemical/enzymatic step is required to remove the cells. This is far too expensive for commercial scale clean meat production and, further, destroys any tissue-like structure. Thus, the present invention contemplates a membrane that is consumed with the meat cells used in the production of a cultured meat product. The present invention further contemplates a membrane that is at least partially dissolvable. This aspect may be needed to, for example, achieve the desired texture to the final structured meat product.

Food-based materials for adherent cell scaffolds have been described in the art. However, these material formats are not suitable for (hollow fiber) membrane bioreactors. These material formats are commonly non-porous films, fiber-based mats (such as electrospun or rotary jet spun), or sponges (usually derived from freeze drying, extrusion processes, and/or foaming processes).

A membrane bioreactor requires a very specific pore size with specific membrane geometries. Hollow fiber bioreactors (HFBRs) typically have a pore size between 5 KDa and 0.1 μm-depending on the cell type, bioreactor design and bioprocess.

Although this invention contemplates hollow fibers, the general concepts of the present invention can be applied to flat sheet (film-like) membranes as well. Sheet membranes are formed, for example, by casting the polymer onto a sacrificial surface which then enters a bath designed for solidification of the polymer. Hollow fibers are formed by being spun out of a nozzle/spinneret into a bath. When producing hollow fibers the bore fluid must also be correctly determined and controlled, as is known to one of skill in the art. Further details about sheet membrane and hollow fiber production follow.

The methods we have invented to generate the membranes of the present invention utilizes multiple steps. For human nutritional considerations and cell adherence considerations, high protein content is preferred. However, the molecular weight of proteins is generally too low to give sufficient chain entanglement or structural integrity for fiber forming properties. Because of this, an additional “carrier” polymer is added to the membrane polymer (i.e., dope solution). As taught herein, the carrier polymer is a polysaccharide, for example, selected from one or more of alginate, cellulose, pectin, chitin, chitosan, gellan gum, xanthan gum, arabinoxylan, glucomannan and others known to one of ordinary skill in the art.

The protein(s) and polysaccharide(s) are mixed in a blend of GRAS solvents. Once one or more proteins and one or more polysaccharides are selected and a mixture thereof formed, they are solidified in a solidification (formation) bath to instantaneously or nearly instantaneously lock in dimensions of the membrane being cast. In an embodiment, it is contemplated that the bath contains multivalent cations such as, for example, Ca2+, Mg2+, or similar. Specifically, demonstrated by the present inventors was that Ca2+ will instantaneously crosslink the alginate, pectin or other polysaccharide in the membrane. This fixes the dimension of the fiber/sheet, achieving the desired 3-dimensional target.

However, at this point, the protein is not crosslinked, and the polysaccharide is only ionically crosslinked. As described in literature, and found in practice, ionically crosslinked polysaccharides can dissociate in cell culture media. Thus, an addition crosslinking step is required to further increase the stability of the membrane and ensure its integrity when use for cell culture. Since harsh chemicals are required for covalent crosslinking, this approach is not preferred for an edible product. The innovation of the present invention is to use physical crosslinking, said physical crosslinking being generated via an energy source such as one or more of heat, gamma, e-beam, beta, x-ray, or UV. These are understood by one of skill in the art to be safe for use in a food product as they are used in the food industry to kill or weaken potential pathogens.

It is further contemplated by the present invention that an alternative approach is to use a crosslinking agent for proteins that is already approved for food use, such as transglutaminase. It is still further contemplated that the polysaccharide(s) may be modified before creating the mixture to increase potential crosslinking sites on the polymer in addition to or in lieu of crosslinking the proteins.

The present invention further contemplates other approaches such as dissolving the protein directly into an alcohol/water blend, and solidifying the membrane in an acid bath. The present invention, still further contemplates dissolving plant protein isolates in alkaline solution then solidifying with organic coagulants like alcohol or a neutralizing acid/caustic solution. For example, if chitosan is dissolved in 5% acetic acid, and extruded into a higher pH bath, the polymer will solidify in the shape of the fiber.

Chitosan can also be dissolved in a slightly acidic bath (about 5% Acetic Acid, citric acid, or similar) then deposited/spun in a bath that contains some concentration of tripolyphosphate/sodium tripolyphosphate (TPP) which will keep and/or maintain the porosity of the solidified chitosan. The bore fluid can also contain a solution similar to the bath solution.

A chemical or enzyme crosslinking agent scan(s) also be added to the bore fluid (fluid used at the nozzle bore when forming solid or hollow fibers; bore fluids are known to one of ordinary skill in the art) and/or formation bath to aid in the crosslinking of the plant proteins that are in the polysaccharide and protein blend. An example of crosslinking agents that may be optionally included in the bath or bore fluid are transglutaminase, tripolyphosphate, genipin (genipin is a chemical compound found in Genipa americana fruit extract), or other oxidative enzymes known to one of ordinary skill in the art.

Another aspect of this invention is that the dope solution (i.e., the protein, polysaccharide mixture) can be impregnated with non-soluble (at least in the solvent system used) fibers. These fibers can be, for example, bacterial nanocellulose, nanocellulose, or other suitable fiber. These fibers can serve two functions, the first being mechanical reinforcement that would result increased “toughness” as defined by stress strain curve charts. The second function of these fibers would be to promote myotube alignment. During extrusion, these fibers naturally align themselves with the hollow fiber and those fibers that are at the surface of the hollow fiber membrane will promote the alignment of cells grown there.

Another aspect of this invention is the geometry and topography of the fiber itself. Preferably, the fiber has an outer diameter of about 300 to about 700 microns. Striations or grooves that run parallel, substantially parallel or essentially parallel with the fiber length can be a structural feature that is desired and built into the fibers made by the methods of the present invention. Striations or grooves along the fiber can be built into the spinning process through the dope solution formulation and mixing, through the nozzle geometry, or through the turbulence of the formation bath by methods known to one of ordinary skill in the art.

It is further contemplated that another step in the process may be increasing cell adherence on the membranes and fibers by using a desired chemical process or compound that alters the surface of the membrane or fibers or coats the membranes or fibers. Examples of suitable processes and compounds include, but are not limited to, plasma treatment, adding cell binding sites through the addition of proteins including but not limited to fibronectin, fibrinogen, laminin, collagen, gelatin, etc., or short peptide sequences isolated from those proteins including but not limited to, RGD, YIGSR, IKVAV, DGEA, PHRSN, PRARI, etc.

Coatings are contemplated that can be applied for target applications beyond cell adhesion as well. Heparin can increase growth factor concentration at the fiber surface. Compounds that will help cell differentiation can also be applied. For example, coatings with high lipid content can promote differentiation of suitable cells into adipocytes

A coating(s) directed toward non-biological (i.e., not directly related to the growth and maintenance of the desired cells) outcomes are also contemplated. Preservatives and/or antibiotics can be used to prevent spoilage or maintain an aseptic environment before and during culture. Dyes, pigments, beta-carotene, etc., can be applied as a coating or directly into the fiber dope solution to give the desired appearance. Similarly, flavor and fragrances can be applied as a coating or directly into the fiber dope solution to give the desired flavor profile. Plasticizers (for example, sugar alcohols such as sorbitol and glycerol) can also be applied as a coating or directly into the dope solution or into the bore fluid. The plasticizer will increase handleability, minimize pore collapse, extend shelf life, as well as alter mouth feel.

The present invention also comprises membranes (hollow fiber and sheet membranes) made by the methods of the present invention.

The present invention contemplates a method for manufacturing cross-linked, edible, porous hollow fibers and membrane sheets, comprising: a) providing: i) one or more edible proteins, ii) one or more edible polysaccharides, iii) one or more solvents and iv) a formation bath, wherein the one or more solvents or the formation bath also comprise one or more multivalent cations or anions; b) co-mixing the one or more edible proteins and one or more edible polysaccharides in the one or more solvents to form a mixture; c) extruding the mixture into the formation bath to form an extruded hollow fibers or casting the mixture onto a bath to form a membrane sheet; and d) exposing the extruded hollow fiber or membrane sheet to an energy source selected from one or more of heat and irradiation sufficient to at least partially crosslink the one or more proteins to form cross-linked, edible, porous hollow fibers.

The present method further contemplates that the one or more proteins are selected from a group consisting of pea, soy, wheat, pumpkin, rice, brown rice, sunflower, canola, chickpea, lentil, mung bean, navy bean, corn, oat, potato, quinoa, sorghum and peanut.

The present method further contemplates that the one or more polysaccharides are selected from a group consisting of agar, chitosan, chitin, alginate, sodium alginate, cellulose, hydroxypropyl cellulose, Methyl cellulose, hydroxypropyl methylcellulose, gellan gum, xanthan gum, pectin, tapioca, guar gum and bean gum.

The present method further contemplates that the one or more solvents are selected from a group consisting of water, acetic acid, citric acid, lactic acid, phosphoric acid, malic acid, tartaric acid, sodium hydroxide, ethanol, glycerin and propylene glycol.

The present method further contemplates that the ion is selected from the group consisting of Ca2+, Mg2+, Fe3+, Zn2+, tripolyphosphate and trisodium citrate and wherein the selected ion is capable of at least enabling partial crosslinking of the one or more polysaccharides.

The present method further contemplates that the heat is from about 120° C. to about 140° C., applied under a pressure of from about 0 PSI to about 20 PSI gauge, at a relative humidity of from about 50% to about 100%, for about 2 to about 60 minutes or the fiber is dipped in a water bath that is from about 60° C. to about 100° C. at atmospheric conditions.

The present method further contemplates that the irradiation is selected from the group consisting of electron beam, UV light and gamma irradiation, that the irradiation is applied in process or post process and that the irradiation is from about 1 to about 100 kGy or from about 10 to about 50 kGy.

The present method further contemplates that the porosity of the hollow fibers or membrane sheets is about 1% to about 90% or from about 50% to about 80%.

The present method further contemplates that the method further comprises coating the cross-linked, edible, porous hollow fiber with a coating to enhance cell adhesion.

The present method further contemplates that the coating is selected from one or more of fibronectin, fibrinogen, laminin, collagen, gelatin or short peptide sequences isolated from those proteins.

The present method further contemplates that the short peptide sequences are selected from the group consisting of RGD, YIGSR, IKVAV, DGEA, PHRSN and PRARI.

The present method further contemplates that the method further comprises modifying the outer surface of the cross-linked, edible, porous hollow fiber to enhance cell adhesion and that the surface modification is selected from one or more of plasma, corona, abrasion, etching, ablation, or sputter coating.

The present method further contemplates that the proteins are powdered or finely milled prior to their dissolution in the solvent.

The present method further contemplates that the proteins are at least 70%, 80%, 90%, 95%, 98%, 99%, 99.9% pure.

The present method further contemplates that the polysaccharides are at least 70%, 80%, 90%, 95%, 98%, 99%, 99.9% pure.

The present method further contemplates that the ratio of protein to polysaccharide is said mixture is from approximately 10:1 to approximately 1:10 or the ratio of protein to polysaccharide in said mixture is approximately 4:1 to approximately 1:4. The present method further contemplates that the ratio of protein to polysaccharide in said mixture is approximately 1:1. The present method further contemplates that the ratio of protein to polysaccharide in said mixture is approximately 1:7 or approximately 7:1. In some cases the solid ratio between protein and polysaccharide are 100:1 or approximately 1:100, or exclusively 100% protein isolate.

The present method further contemplates that the formation bath comprises, for example, RO (reverse osmosis) water with dissolved calcium chloride at or approximately at the concentration of 15 g/L, however, the desired concentration may be from about 4 g/L to about 20 g/L, about 12 g/l to about 18 g/L or about 14 g/L to about 16 g/L. In a continuous process, the formation bath will have a feed and bleed system, where prepared 15 g/L calcium chloride is fed into a side of the bath, and where the bath is bled at the same rate.

The present method further contemplates that the formation bath comprises RO water with one or more of calcium, zinc, magnesium, iron and potassium, in combination with one or more of i) water, acetic acid, citric acid, lactic acid, phosphoric acid, malic acid, tartaric acid, or one or more of ii) sodium hydroxide and potassium hydroxide.

The present method contemplates that a method for manufacturing cross-linked, edible, porous hollow fibers and membrane sheets, comprising: a) providing: i) one or more edible proteins, ii) one or more edible polysaccharides, iii) one or more solvents and iv) a formation bath, wherein the formation bath is predominantly water and further comprises one or more of calcium chloride, zinc chloride, magnesium ions, potassium, in combination with 1) one or more of acetic acid, citric acid, lactic acid, phosphoric acid, malic acid, tartaric acid or other suitable acid, or 2) one or more of sodium hydroxide and potassium hydroxide or other suitable base; b) co-mixing the one or more edible proteins and one or more edible polysaccharides in the one or more solvents to form a mixture; c) extruding the mixture into the formation bath to form an extruded hollow fibers or casting the mixture onto a bath to form membrane sheets; and d) exposing the extruded hollow fiber or membrane sheet to an energy source selected from one or more of heat and irradiation sufficient to at least partially crosslink the one or more proteins to form cross-linked, edible, porous hollow fibers. In this embodiment, the formation bath is supplemented with ions.

The present method further relates to and contemplates any hollow fiber or sheet membrane (i.e., membrane sheet) that is made by the methods of the present invention.

The present invention further relates to clean meat, structured meat, cultured meat, lab grown meat, cultivated meat, cell-based meat, or the like, produced with the membranes or the present invention, and methods for making the same.

It is contemplated that the present invention relates to a method for manufacturing cross-linked, edible, porous hollow fibers or sheet membranes, the method comprising: a) providing: i) one or more edible proteins, ii) one or more solvents iii) a formation bath; wherein the one or more solvents or the formation bath also comprise one or more multivalent cations or anions or a buffer solution; b) co-mixing the one or more edible proteins in the one or more solvents to form a mixture; c) extruding the mixture into the formation bath to form an extruded hollow fiber or casting the mixture into the formation bath to form a sheet membrane; and d) exposing the extruded hollow fiber or sheet membrane to an energy source selected from one or more of heat and irradiation sufficient to at least partially crosslink the one or more proteins to form cross-linked, edible, porous hollow fibers or sheet membrane.

It is further contemplated that the methods of the present invention relate to providing one or more edible polysaccharides and co-mixing the one or more polysaccharides with the one or more edible proteins in the one or more solvents.

It is further contemplated that the methods of the present invention relate to providing a plasticizer and co-mixing the plasticizer with the one or more edible proteins in the one or more solvents.

It is further contemplated that the methods of the present invention relate to wherein the one or more proteins are selected from a group consisting of pea, soybean, wheat, pumpkin, rice, brown rice, sunflower, canola, chickpea, lentil, mung bean, navy bean, corn, oat, potato, quinoa, sorghum and peanut.

It is further contemplated that the methods of the present invention relate to wherein the one or more polysaccharides are selected from a group consisting of agar, chitosan, chitin, alginate, sodium alginate, cellulose, hydroxypropyl cellulose, Methyl cellulose, hydroxypropyl methylcellulose, gellan gum, xanthan gum, pectin, tapioca, guar gum and bean gum.

It is further contemplated that the methods of the present invention relate to the one or more solvents are selected from a group consisting of water, acetic acid, citric acid, lactic acid, phosphoric acid, malic acid, tartaric acid, sodium hydroxide, ethanol, glycerin and propylene glycol.

It is further contemplated that the methods of the present invention relate to wherein the formation bath comprises one or more of calcium, zinc, magnesium, iron and potassium, in combination with one or more of 1) water, acetic acid, citric acid, lactic acid, phosphoric acid, malic acid, tartaric acid, or one or more of 2) sodium hydroxide and potassium hydroxide.

It is further contemplated that the methods of the present invention relate to wherein said ion is selected from the group consisting of Ca2+, Mg2+, Fe3+, Zn2+, tripolyphosphate and trisodium citrate and wherein said selected ion is capable of at least enabling partial crosslinking of the one or more polysaccharides.

It is further contemplated that the methods of the present invention relate to wherein the mixture of step b) is heated.

It is further contemplated that the methods of the present invention relate to wherein said formed hollow fiber or sheet membrane is heated from about 70° C. to about 140° C. or from about 120° C. to 140° C., applied under a pressure of from about 0 PSI to about 20 PSI gauge, at a relative humidity of from about 50% to about 100%, for about 2 to about 60 minutes or the hollow fiber or sheet membrane is dipped in a water bath that is from about 60° C. to about 100° C. at atmospheric conditions.

It is further contemplated that the methods of the present invention relate to wherein the co-mixing is performed at about 0° C. to about 90° C.

It is further contemplated that the methods of the present invention relate to wherein said mixture is at a pH of about 10 to about 13 and said formulation bath is at a pH of about 3 to about 5.

It is further contemplated that the methods of the present invention relate to wherein after formation, the membrane is neutralized to a pH of about 6.8 to about 7.8.

It is further contemplated that the methods of the present invention relate to wherein after formation, the membrane is neutralized to a pH of about 7.3 to about 7.5.

It is further contemplated that the methods of the present invention relate to wherein the irradiation is selected from the group consisting of electron beam, UV light and gamma irradiation.

It is further contemplated that the methods of the present invention relate to wherein the irradiation is applied in process or post process. It is further contemplated that the methods of the present invention relate to wherein the irradiation is from about 1 to about 100 kGy or from about 10 to about 50 kGy.

It is further contemplated that the methods of the present invention relate to wherein the porosity of the hollow fiber or sheet membrane is from about 1% to about 90%, about 25% to about 75% or about 40% to about 60%.

It is further contemplated that the methods of the present invention relate to wherein the porosity of the hollow fiber or sheet membrane is from about 50% to about 80%.

It is further contemplated that the methods further comprise coating the cross-linked, edible, porous hollow fiber or sheet membrane with a coating to enhance cell adhesion.

It is further contemplated that the methods of the present invention relate to wherein the coating is selected from one or more of fibronectin, fibrinogen, laminin, collagen, gelatin or short peptide sequences isolated from those proteins.

It is further contemplated that the methods of the present invention relate to wherein the short peptide sequences are one or more selected from the group consisting of RGD, YIGSR, IKVAV, DGEA, PHRSN and PRARI.

It is further contemplated that the methods of the present invention relate to modifying the outer surface of the cross-linked, edible, porous hollow fiber to enhance cell adhesion. It is further contemplated that the present invention relates to the method further comprising coating the cross-linked, edible, porous hollow fiber or sheet membrane with a plasticizer. It is further contemplated that the present invention relates to wherein the surface modification is selected from one or more of plasma, corona, abrasion, etching, ablation, or sputter coating.

It is further contemplated that the methods of the present invention relate to wherein the proteins are powdered or finely milled prior to their dissolution in the solvent.

It is further contemplated that the methods of the present invention relate to wherein the proteins are at least 70%, 80%, 90%, 95%, 98%, 99%, 99.9% pure.

It is further contemplated that the methods of the present invention relate to wherein the polysaccharides are at least 70%, 80%, 90%, 95%, 98%, 99%, 99.9% pure.

It is further contemplated that the methods of the present invention relate to wherein the ratio of protein to polysaccharide (protein: polysaccharide) in said mixture is from approximately 10:1 to approximately 1:10 or approximately 1:99 to approximately 99:1, 98:2, 97:3, 96:4, 95:5 or 90:10. It is further contemplated that the present invention relates to wherein the ratio of protein to polysaccharide in said mixture is approximately 4:1 to 1:4. It is further contemplated that the present invention relates to wherein the ratio of protein to polysaccharide in said mixture is approximately 1:1 or 7:1.

It is further contemplated that the present invention relates to wherein the formation bath comprises one or more of calcium, zinc, magnesium, iron and potassium, in combination with one or more of i) water, acetic acid, citric acid, lactic acid, phosphoric acid, malic acid, tartaric acid, or one or more of ii) sodium hydroxide and potassium hydroxide.

It is further contemplated that the present invention relates to a hollow fiber or sheet membrane made by any of the methods of the present invention.

It is contemplated that the present invention relates to a method for manufacturing cross-linked, edible, porous hollow fibers or sheet membranes, comprising: a) providing: i) one or more edible proteins, ii) one or more edible polysaccharides, iii) one or more solvents and iv) a formation bath, wherein the formation bath comprises one or more of calcium, zinc, magnesium, iron and potassium, in combination with one or more of 1) water, acetic acid, citric acid, lactic acid, phosphoric acid, malic acid, tartaric acid, or one or more of 2) sodium hydroxide and potassium hydroxide; b) co-mixing the one or more edible proteins and one or more edible polysaccharides in the one or more solvents to form a mixture; c) extruding the mixture into the formation bath to form an extruded hollow fibers or casting the mixture to form a sheet membrane; and d) exposing the extruded hollow fiber or sheet membrane to an energy source selected from one or more of heat and irradiation sufficient to at least partially crosslink the one or more proteins to form cross-linked, edible, porous hollow fibers.

It is contemplated that the present invention relates to methods for the manufacture of hollow fibers or sheet membranes wherein one or more proteins, one or more polysaccharides, one or more solvents, plasticizer(s) and/or one or more constituents of the formation bath is generally recognized as safe (GRAS) by the U.S. Food and Drug Administration (FDA).

It is further contemplated that the present invention relates to the resulting membrane or hollow fiber made by any of the methods of the present invention undergoes a 10-50% glycerol in water exchange for drying and said drying does not result in pore collapse.

### BRIEF DESCRIPTION OF THE FIGURES

FIG. 1 shows a schematic diagram of one process used to produce the membranes and hollow fibers of the present invention.

FIG. 2 shows a schematic diagram of another process used to produce the membranes and hollow fibers of the present invention.

FIGS. 3(A & B) shows hollow fiber membranes produced with the methods of the present invention.

FIG. 4(A-C) shows scanning electron micrographs (SEM) of fibers produced with the methods of the present invention. A shows surface pores of whey protein and alginate blend can be seen to be approximately 20 nm or about 1000 kDa. This image also shows the striations from the process are parallel with the length of the fiber. B shows surface pores of a pumpkin protein isolate and alginate blend having surface pores of approximately 100 nm and smaller. C. shows a lower resolution image of the fiber made with pumpkin protein isolate.

FIGS. 5A & B show a fiber manufactured by the methods of the present invention. The hollow fibers of the present invention can easily support their weight, which will be needed in a bioreactor. (A) The fiber shown is 2 meters long. (B) The fiber produced by the methods of the present invention can support at least 9 grams.

FIG. 6 shows mung bean casted film out of a urea and sodium hydroxide solution. Image is of the mung bean dope solution cast onto glass via doctoral blade technique. It can be seen that the dope solution is transparent prior to coagulation.

FIG. 7 shows viscosity using a Brookfield (Middleboro, MA) viscometer equipped with a S64 spindle the viscosity of 2% alginate and 10% protein isolates are displayed. Each mix had the pH adjusted to 11 pH prior to measurement.

FIG. 8 shows simple design plot in amounts. This is a design of experiments using Minitab (State College, PA) looking at urea, ethanol, and water with sodium hydroxide.

FIG. 9 shows a temperature sweep of 15% zein in the solvent blends form FIG. 8. Which shows that that solvents systems with as low as 12.5% ethanol can dissolve zein.

FIG. 10 shows that by using a solvent condition from FIG. 8, the gelation properties of agarose can be altered; when compared to the same agarose in water.

FIG. 11 shows that within a given mixing temperature ranges, Zein and agarose can blended without solidification of either component with a given solvent system from FIG. 8; especially above 40° C.

FIGS. 12A & B shows an image of the zein membrane production process consisting of a film casting step (A: left) and coagulation step in acetate buffer (0.2 M, pH 4.5) (B: right).

FIG. 13 shows the crosslinking step of a mung-bean alginate membrane using a hot glycerol bath set at 120° C., over 1 hour.

FIGS. 14A & B shows graphs showing the elastic moduli (A: left) and strains of membranes (B: right).

FIGS. 15A & B shows the elastic moduli of various tissues (A: left) and exemplary membrane materials of the present invention (B: right), respectively.

FIG. 16 shows images (1-6) of the membranes produced according to different manufacturing protocols to explore and validate each production steps. AC stands for “acetate bath 0.2 M at pH 4.5”, H stands for “HEPES Buffer”+70+50k|gur{|hwk|o040 slshud}lqhhwkdqhvxarqlf dflg, bath 0.1 M at pH 7.4, G stands for “glycerol bath”, HG stands for “hot glycerol bath”, HW stands for “hot water” (autoclave) and 0 stands for “step not performed”.

FIGS. 17A & B shows the elastic moduli (A: left) and strain to break (B: right) of membranes, respectively. Sample 6 is produced according to protocol AC-O-G-HG, 5 according to protocol AC-O-O-HG and 2 according to protocol AC-O-O-HW.

FIG. 18A-C shows the change in elastic modulus (A; left), strain (B: center) and final stress (C: right) upon increase of coagulation time in acetic bath for thermally treated (glycerol-based protocol) mung bean membranes. The figure shows the mechanical properties of the membranes which were coagulated for 10 minutes up to 3 hours.

FIG. 19A-C shows the change in elastic modulus (A: left), strain (B: center) and final stress (C: right) upon increase of glycerol-based thermal treatment time for mung bean membranes. The glycerol-based heat treatment was investigated by keeping constant the duration of both coagulation bath (10 mins) and water-glycerol exchange (10 mins) and varying the heat treatment duration after reaching the final temperature of 120° C.

FIG. 20 shows Rheology investigation on the heat treatment of mung bean membranes using glycerol. Graph showing the variation of Tan Delta (δ) over a temperature gradient.

FIG. 21A-C shows the change in A) elastic modulus, B) strain and C) final stress upon increase of glycerol-based thermal treatment time for mung bean membranes.

FIG. 22 shows the elastic modulus values for alginate and gluten protein blends, including wheat gluten, mung bean and zein, when incubated at 37° C. cell-media. Mechanical tensile measurements were taken before and after 3, 10, and 21 days of incubation.

FIG. 23 shows the strain to break values for alginate and gluten protein blends, including wheat gluten, mung bean and zein, when incubated at 37° C. cell-media. Mechanical tensile measurements were taken before and after 3, 10, and 21 days of incubation.

FIG. 24 shows membrane surface area for alginate protein blends, including wheat gluten, mung bean and zein, when incubated at 37° C. cell-media. Measurements were taken before and after 3, 10, and 21 days of incubation.

FIG. 25 shows membrane surface area for agarose protein blends, including wheat gluten, mung bean and zein, when incubated at 37° C. in cell-media. Measurements were taken before and after 3, 10, and 21 days of incubation.

FIGS. 26A & B shows the comparison in A) elastic modulus and B) strain between the brown rice-alginate blends prepared with and without transglutaminase crosslinking, before and after 3, 10 and 21 days of incubation at 37° C. in cell media.

FIG. 27A-F shows the elastic modulus (A & D: left), strain to break values (B & E: center) and surface area (C & F: right) of protein membranes including soy protein isolate (A-C: top) and mung bean (D-F: bottom). Measurements were taken before and after 3, 10 and 21 days of incubation in cell media at 37° C. for the soy protein isolate and before and after 5, 12 and 30 days of incubation cell media at 37° C. for mung bean.

FIG. 28 shows scanning electron microscopy images of soy protein isolate membrane surface (top) and cross section (bottom).

FIG. 29 shows scanning electron microscopy images of mung bean protein isolate membrane surface (top) and cross section (bottom).

FIG. 30 shows scanning electron microscopy images of zein protein isolate membrane surface (top) and cross section (bottom) and zein protein isolate & agarose membrane surface (top) and cross section (bottom).

FIG. 31 shows scanning electron microscopy images of the surface and cross section of zein-alginate (left) and pea protein-k-carrageenan (right) membrane.

FIG. 32 shows scanning electron microscopy images of the surface and cross section of mung bean-agarose (left) and soy-alginate (right) membranes.

FIG. 33 shows scanning electron microscopy of a mung bean-alginate hollow fiber cross section (top) and surface (bottom).

FIG. 34 shows fluorescent cell adhesion and proliferation studies carried out on zein, soy, mung bean TG-crosslinked mung bean membranes, using the C2C12 cell line. Live (green)/dead (red) assay carried out after 48 hours of growth period. Micrographs reveal nearly no red staining indicating that nearly all cells are alive.

FIG. 35 shows cell fluorescent adhesion and proliferation studies carried out on fibronectin-, collagen- and chitosan-coated mung bean membranes and chitosan membranes, using the C2C12 cell line. Live (green)/dead (red) assay carried out after 48 hours of growth period. Micrographs reveal nearly no red staining indicating that nearly all cells are alive.

FIG. 36 shows fluorescent cell adhesion and proliferation studies carried out on thermally treated and non-thermally treated soy-alginate, peanut-alginate and zein-agarose membranes, using the C2C12 cell line. Live (green)/dead (red) assay carried out after 48 hours of growth period. Micrographs reveal nearly no red staining indicating that nearly all cells are alive.

FIG. 37 shows fluorescent cell adhesion and proliferation studies carried out on soy, fibronectin- and collagen-coated mung bean and chitosan membranes, using the QM7 cell line. Live (green)/dead (red) assay carried out after 48 hours of growth period. Micrographs reveal nearly no red staining indicating that nearly all cells are alive.

FIG. 38 shows the effects of drying and rehydration on alginate: mung bean-based membrane.

### DETAILED DESCRIPTION OF THE INVENTION

### Structured Meat Products

The present invention contemplates edible membranes including, but not limited to, hollow fibers of suitable integrity for use in bioreactors for the production, for example, of structured clean meat, and methods of production of structured clean meat therewith and the structured clean meat produced with the hollow fibers of the present invention. Clean meat (also known in the art as “cultured meat” or “lab grown meat”) is defined in the art as meat or a meat-like product (referred to collectively herein as “clean meat” or “clean meat product”) grown from cells in a laboratory, factory or other production facility suitable for the large-scale culture of cells.

A “structured meat product,” “structured clean meat product,” “structured cultured meat” or “structured cultured meat product” is a meat product or clean meat product having a texture and structure like, similar to or suggestive of natural meat from animals. The structured meat product of the present invention has a texture and structure that resembles natural meat 1) in texture and appearance, 2) in handleability when being prepared for cooking and consumption (e.g., when being sliced, ground, cooked, etc.) and 3) in mouth feel when consumed by a person. The materials and methods of the present invention, when used in the production of structured clean meat, achieve at least one of these criteria, two of these criteria or all three of these criteria. The prior art technology is unable to produce a structured meat product sufficiently meeting any of these criteria.

The structured meat product of the present invention meets these criteria by culturing suitable cells (discussed, infra) in a bioreactor (also, discussed, infra) comprising the hollow fibers of the present invention. The hollow fibers of the present invention, at least in considerable part, provide the structure and texture to the final structured clean meat product that provides the desired appearance, handleability and mouth feel of the product. Further, the hollow fibers of the present invention aid in providing a suitable environment for the growth of the cells into a structured clean meat product. In this context, the hollow fibers of the present invention provide at least a surface suitable for the attachment of the cultured cells, elongation of the cells into morphologies resembling myocytes or myocyte-like cells (i.e., substantially resembling myocytes in structure and appearance), and formation of the myocytes into myotubule or myotubule-like structures (i.e., substantially resembling myotubules in structure and appearance).

### Production of Membranes of the Present Invention

It is understood that in the present invention the term “membrane” or “membranes” refers to any porous membrane structure produced by the methods of the present invention including, but not limited to, hollow fiber membranes and sheet (i.e., flat) membranes. Unless specifically indicated otherwise, reference to “membranes,” “hollow fibers,” “hollow fiber membranes” and “sheet membranes” will be understood to inclusive of any membrane structure produced by the methods of the present invention regardless of shape, form or appearance.

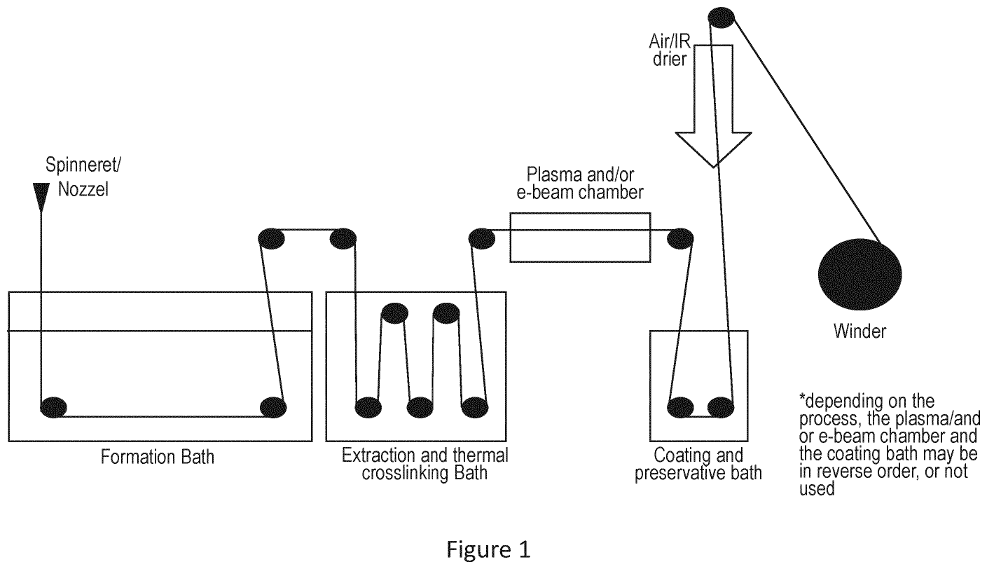

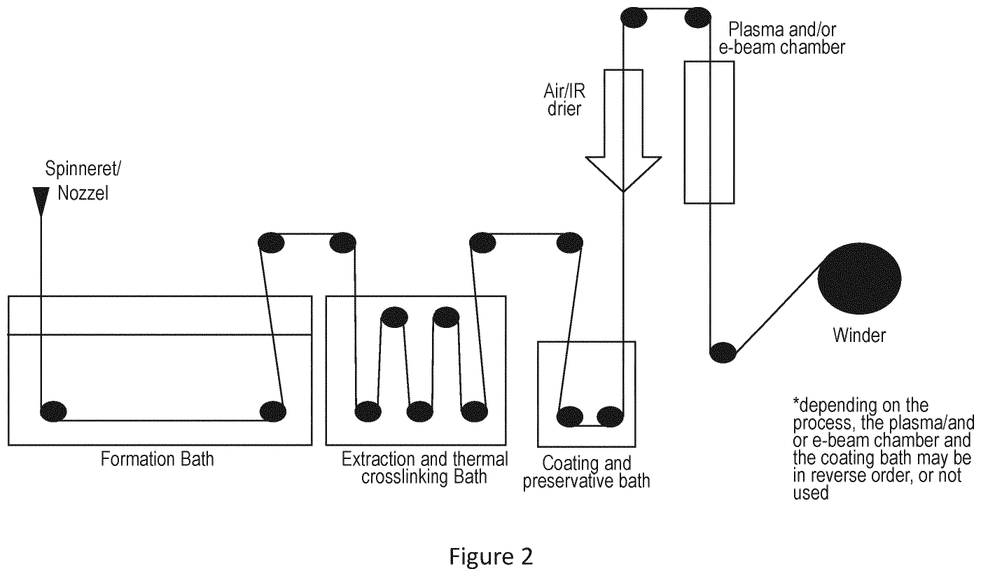

Exemplary production processes are shown in schematic form in FIGS. 1 & 2.

It is contemplated that the edible and/or dissolvable hollow fibers and sheet membranes of the present invention are made from one or more of hydrocolloids (i.e., polysaccharides such as Xanthan, methyl cellulose(s), alginate, agar, pectin, gelatin, carrageenan, cellulose/gellan/guar/tara/bean/other gums), proteins (e.g., polypeptides, peptides, glycoprotein and amino acids; for example, various starches (corn/potato/rice/wheat/sorghum), plant isolates (e.g., soy/zein/casein/wheat/mung protein), lipids, (e.g., free fatty acids, triglycerides, natural waxes, and phospholipids), alcohols (e.g., polyalcohol), carbohydrates and other natural substances such as alginate. Further, it is contemplated that other materials may be added to the hollow fibers or coated on to the hollow fibers that aid in cell attachment and cell growth. For example, it is contemplated that the hollow fiber additive or coating is one or more of proteins, hydrogels, or other coatings known by one of skill in the art including extra cellular matrix (ECM) components and extracts, poly-D-lysine, laminin, collagen (e.g., collagen I and collagen IV), gelatin, fibronectin, plant-based ECM materials, collagen-like, fibronectin-like and laminin-like materials known to one of ordinary skill in the art that are isolated from a plant or synthesized from more simple substances. The overall result is that the fibers of the present invention impart the texture and structure of meat and meat products giving the structured clean meat product produced by the present invention a texture, appearance, handleability and mouth feel similar to real meat.

It is noted by the Inventors of the present invention that soy and mung bean protein isolates confer several of the desired characteristics to the membranes produced by the methods of the present invention. It is also noted by the Inventors that both soybean (Glycine max) and mung bean (Vigna radiata) are from the same classification family related to legumes (i.e., peas or beans), Fabaceae. Doyle, J. J., Leguminosae, Encyclopedia of Genetics, 2001, 1081-1085. Although the present invention is not limited by theory, it is believed that other members of this family, especially the Millettioids and Phaseoloids including the geneses Glycine and Vigna, will work substantially similar to soy and mung bean protein isolates. See, FIG. 39.

More specifically, the hollow fibers of the present invention may comprise one or more of cellulose, chitosan, collagen, zein, alginate, agar, inulin, gluten, pectin, legume protein, methyl cellulose(s), gelatin, tapioca, xanthan/guar/tara/bean/other gums, proteins (e.g., polypeptides, peptides, glycoprotein and amino acids including, but not limited to, various forms of corn/potato/rice/wheat/sorghum starches, plant isolates and soy/zein/casein/wheat protein, all of which are known to one of skill in the art), lipids, (for example, free fatty acids, triglycerides, natural waxes, and phospholipids). Cellulosic polymers may include cellulose acetate-butyrate, cellulose propionate, ethyl cellulose, methyl cellulose, nitrocellulose, etc. More specifically, the hollow fibers of the present invention may comprise a mixture of one or more legume proteins and hydrocolloids.

In an embodiment, it is contemplated that the hollow fibers of the present invention are edible, dissolvable or edible and dissolvable. In other words, the fibers may be either edible or dissolvable or both. Further still, for fibers that are dissolvable, there may be differing degrees of dissolvability. For example, some fibers may be readily dissolvable upon exposure to a suitable solvent (e.g., a non-toxic solvent that is generally recognized as safe by the Food and Drug Administration (FDA) or other organization recognized as being qualified to assess the safety of consumable substances). Other fibers may be less readily dissolvable. In this regard, the less readily dissolvable fibers may be partly dissolved after the cells being cultured have reached the requisite level of confluency thereby leaving enough of the fiber to provide for a desired mouth feel and texture to the structured clean meat of the present invention but not an excess of fiber that may make the structured clean meat product of the present invention seem tough or chewy. Dissolvable hollow fiber constituents are known to those of skill in the art. For example, alginate is dissolvable upon exposure to a Ca2+ chelator. In an embodiment of the present invention, it is contemplated that the hollow fibers of the present invention comprise an amount of alginate to render the fibers partially dissolvable and/or a percentage of fibers in a device comprising the hollow fibers of the present invention comprise alginate.

In an embodiment of the present invention, it is contemplated that one or more crosslinkers are used in the hollow fibers of the present invention. Crosslinkers, as the name implies, bind one or more of the other constituents of the hollow fiber to strengthen the fiber. In an embodiment of the present invention, the crosslinker may be the dissolvable component or one of the dissolvable components of the hollow fibers of the present invention. Exemplary crosslinkers and crosslinking mechanisms as contemplated by the present invention, include but are not limited to, covalently bonded ester crosslinks (U.S. Pat. No. 7,247,191) and UV-crosslinking (U.S. Pat. No. 8,337,598), both of which are incorporated herein by reference in their entirety. Further, use of crosslinkers in the production of hollow fibers is known to one of skill in the art. See, for example, U.S. Pat. Nos. 9,718,031; 8,337,598; 7,247,191; 6,932,859 and 6,755,900, all of which are incorporated herein in their entirety.

The membranes and fibers of the present invention are produced from a blend of protein(s) and polysaccharide(s). The ratio of protein to polysaccharide is contemplated to be from approximately 1:99 to approximately 99:1, approximately 1:10 to 10:1, approximately 2:5 to 5:2, approximately 3:7 to 7:3, approximately 4:6 to 6:4 or approximately 1:1, or any ratio within the stated rations. In a preferred embodiment, the protein content of the mixture is higher than the polysaccharide content. In a preferred embodiment, the protein content is about 90%, 95%, 98%, 99% or greater.

It is further contemplated that the membranes of the present invention are further strengthened, i.e., given increased integrity and strength, but incorporation of manufacturing process steps that cross-link the proteins in the membrane. The present inventors found that after formation of the membranes of the present invention, if they are exposed to an energy source for an appropriate amount of time are at an appropriate level of energy, the proteins will at least partially crosslink and thereby give the membranes of the present invention increased integrity over membranes of the prior art. The Exemplification section that follows provides examples of several membranes (i.e., hollow fiber membranes) that are processed with and without heat or irradiation. The hollow fibers produced without the addition of being exposed to the stated energy source lacked integrity as compared to those produce with the addition of being exposed to an energy source.

Heat may be supplied via either dry or wet heat. One process of the present invention utilizes a temperature of from about 60° C. to about 100° C. at a pressure of 0 psi (ambient pressure) to 20 psi or greater with a relative humidity of about 50% to 100% and for about 2 to about 60 minutes. Further, heat may be supplied via dipping the membranes or fibers of the present invention into a water bath from about 60° C. to about 100° C. at atmospheric conditions.

The membranes and fibers of the present invention may also be exposed to energy via any form of radiation (e.g., electronic beam, gamma, UV, etc.). The membranes and fibers of the present invention may be irradiated from about 1 to about 100 kGy, from about 5 kGy to about 75 kGy or from about 10 kGy to about 50 kGy. The membranes and fibers of the present invention may be exposed to said radiation from about 0.1 minutes to about 60 minutes, form about 1 minute to about 50 minutes, from about 2 minutes to about 40 minutes, and from about 2 minutes to about 30 minutes, and any value falling within the recited values.

Hollow fiber manufacturing techniques, in particular, and membrane manufacturing techniques, in general, are known to one of skill in the art. (See, for example, Vandekar, V. D., Manufacturing of Hollow Fiber Membrane, Int'l J Sci & Res, 2015, 4:9, pp. 1990-1994, and references cited therein). Like flat sheet membrane, known methods of hollow fiber manufacturing typically include some technique of phase separation. Common methods nonsolvent induced phase separation include thermally induced phase separation, vapor induced phase separation, heat induced phase separation (see, for example U.S. Pat. No. 5,444,097 to MilliporeSigma, which is incorporated herein by reference), or a combination thereof. However, other techniques like thermal extrusion and stretching can be used for hollow fiber and membrane formation. Typically, one would destabilize the polymer in solution by means of nonsolvent, thermal destabilization, or removal of the solvent. As described in here, dissolutions of the polymer (polysaccharides and proteins in this case) will be followed by the gelation or solidification via multiple crosslinking processes. The fibers may be further stretched to produce fibers with diameters less than 100 μm and a wall thickness as thin as 10 μm.

Membrane sheets can be manufactured using similar phase inversion where a liquid polymer solution is solidified as it enters a quenching solution and solvents are drawn out, as well as other techniques known to one of ordinary skill in the art (see, for example, U.S. Patent Publication No. 2020/0368696 to MilliporeSigma) such as but not limited to solvent evaporation. See, for example, Gas Separation Membranes, Polymeric and Inorganic, Chapter 4, Ismail, et al., Springer, 2015 and U.S. Patent Publication No. 2007/0084788 to MilliporeSigma.

In some aspects of the present invention, pH induced phase separation (“pH Induced Phase Separation” or “Proton Induced Phase Separation;” Satoru Tokutomi, Kazuo Ohki, Shun-ichi, Ohnishi, Proton-induced phase separation in phosphatidylserine/phosphatidylcholine membranes, Biochimica et Biophysica Acta (BBA), Biomembranes, Volume 596, Issue 2, Feb. 28, 1980, Pages 192-200.) is used in the manufacture of the membranes (i.e., hollow fibers and sheet membranes) of the present invention. pH induced phase separation is exemplified in the Examples section, infra. While liquid phase separation of macromolecules controlled by pH is studied in cellular physiology (Adame-Arana, O., et al., Liquid Phase Separation Controlled by pH, 2020 Oct. 20; 119 (8): 1590-1605; Epub 2020 Sep. 16) it is believed that the present inventors are the first to utilize pH induced phase separation in the production of hollow fiber and sheet membranes and especially membranes that are suitable for the production of a clean meat or clean structured meat product. The use of pH induced phase separation confers unexpected and surprising benefits on the membranes of the present invention. Namely mechanical integrity, pore size, and porosity are enhanced over conventional processes.

Dry spinning involves dissolving the polymer in a very volatile solvent. The solvent/polymer mixture is heated after extrusion and evaporation of the solvent the polymer solidifies.

Wet spinning is more versatile since the process involves a larger number of parameters that can be varied. The polymer and solvent mixture is extruded into a nonsolvent bath where demixing and/or phase separation occurs because of the exchange of solvent and nonsolvent. Between the extrusion and the nonsolvent bath there is an air gap where the hollow fiber membrane formation begins.

A technique that can eliminate or minimize the use of solvents is melt spinning with cold stretching (MSCS). This approach leads to cost effective production, but may sacrifice structure control and potential degradation of the food materials. In this technique the materials are heated for extrusion and then pulled as they are cooled as to mechanically form pores in the hollow fiber wall. All of three of these techniques have been widely studied and are known in the art they well summarized (see, Tan, X M. and Rodrigue, D., Polymers (Basel), Aug. 5, 2019: 11 (8)).

Modifications of these techniques are also known to one of skill in the art. See, for example, WO 2011/108929 (incorporated herein by reference in its entirety) where a modified wet spinning extrusion process for the production of hollow fibers comprised of multiple polymers and polymer layers is disclosed. Manufacture of hollow fibers from non-synthetic materials is also known to one of skill in the art. See, for example, U.S. Pat. No. 4,824,569 to Suzuki, which is incorporated herein in its entirety.

### Hollow Fiber Membranes of the Present Invention for the Production of a Structured Meat Product

The macroscopic structure of the hollow fibers of the present invention, in an embodiment, is contemplated to promote the orientation of the cells along the fibers. In this regard, it is desired by the present invention that the orientation of the component molecules from which the hollow fiber is constructed be oriented parallel, essentially parallel or predominately parallel to the length of the hollow fibers. In is further contemplated that the component molecules create a surface texture at least on the outer surface of the hollow fiber that aids in cell attachment and aids in cell orientation. Thus, in an embodiment, it is contemplated that the surface texture of the hollow fibers of the present invention create attachment points for cell attachment. In another embodiment, it is further contemplated that the cells grown on the hollow fibers of the present invention (in particular, the myocytes, myocyte-like cells or cells having characteristics of myocytes) orient and extend along the length of the hollow fiber similar to and resembling myocytes in vivo.

Thus, the orientation of the surface structure of the scaffold directly correlates to the alignment of the myotubes during formation. It can be thought of as if skeletal muscle wants to form along a preexisting structure. It can be envisioned that a bundle of fibers closely mimics skeletal muscle structure for the formation of aligned myotubes. Therefore, a hollow fiber bioreactor doesn't only achieve the tissue-like cell densities, but it also achieves the myotube alignment that other technologies do not, resulting in the most realistic mouth feel of all discussed technologies. The alignment phenomena can be better understood by reviewing: My mistake: Decellularized Apium graveolens Scaffold for Cell Culture and Guided Alignment of C2C12 Murine Myoblast-Santiago Campuzano, 2020, Ph.D. thesis, University or Ottawa, pp 58-59.

With regard to producing a structured clean meat product, it is contemplated that the hollow fibers of the present invention have a range of sizes over which they will be suitable for the present invention. It is also contemplated that the hollow fibers of the present invention are spaced such that the cells grown on the hollow fibers achieve a density similar to that of real meat and with a minimum of void space between the cells. In one embodiment, it is contemplated that the hollow fibers of the present invention have an outer diameter of about 0.1 mm to about 3.0 mm, a porosity of about 0% porosity (making it diffusion based) to about 75%, and a wall thickness of about 0.008 to about 0.5 mm or about 0.01 mm to about 0.2 mm or any thickness between 0.008 mm to 0.5 mm not specifically iterated above. It was found by the present inventors that this size is suitable for the transport of media through the lumen of the fiber and permit the adequate flow of media through the wall of the hollow fiber while at the same time being rigid enough to support cell growth and, further, provide for the desired final product structure, texture, handleability and mouth feel. However, depending on the desired structured clean meat product (e.g., beef, poultry, fish, pork, etc.) other embodiments with regard to variations of the diameter, wall thickness and porosity of the fibers are contemplated; discussed infra.

Fiber porosity. The hollow fibers of the present invention need to have a porosity that allows for adequate flow of media though the wall of the fiber while at the same time ensuring a suitable surface for cell growth and cell support. The porosity of the hollow fibers is related, in part, to the thickness of the wall of the hollow fiber and to the composition of the hollow fiber. If the wall is thin enough, then about 0% porosity may suffice allowing the media diffusing through the hollow fiber wall. The porosity of the hollow fibers of the present invention may be as high as 75% or 90%. Thus, the range of porosity of the hollow fibers of the present invention is from 0% to about 90%, from about 10% to about 75%, from about 30% to about 60%, or any percentage value between 0% and 75% not specifically iterated above.

The hollow fibers of the present invention may also be subject to a pore forming step. The pore forming mechanism will be one of the following techniques, well known in the art of membrane formation: TIPS=thermally induced phase separation, NIPS=nonsolvent induced phase separation, VIPS=vapor-induced phase separation, pH induced phase separation, MSCS=melt-spinning combined with stretching, (see, Review on Porous Polymeric Membrane Preparation. Part II: Production Techniques with Polyethylene, Polydimethylsiloxane, Polypropylene, Polyimide, and Polytetrafluoroethylene, Xue Mei Tan, 1, 2, 2019). In all scenarios the polymer will be in a liquid phase either by thermally melting it or chemical dissolution. From there, the polymer is extruded into a cylindrical shape, and drawn onto a spindle. During the extrusion step, a bore fluid can be used to prevent the hollow fiber form collapsing on itself. Between the extruding nozzle and the rewind spindle, there may also be a pore forming chamber, such as a water bath or an atmospheric environmental chamber.

The present invention also contemplates the configuration of the hollow fibers of the present invention in a bioreactor. Fiber configuration may include one or both of fiber positioning and spacing. Fibers may be configured in any configuration that permits growth of the cell population with a minimum of void space between cells at confluency. For example, the fibers can be oriented in square/rectangle (rows and columns) or triangle/hexagonal (honeycomb) packing modes. Thus, in one embodiment it is contemplated that the fibers are arranged such that the fibers, when viewed on end, form an ordered pattern of rows and columns. In another embodiment, it is contemplated that the fibers, when viewed on end, form a honeycomb pattern. In another embodiment, it is contemplated that the fibers of the present invention are arranged randomly or semi-randomly. In another embodiment, it is contemplated that the hollow fibers are arranged in an ordered or semi-ordered pattern of varying densities.

The hollow fibers can range from about 0.1 mm to about 3.0 mm, about 0.5 mm to about 2.0 mm and about 0.8 mm to about 1.3 mm in outer diameter, and any value in between the cited values. A 1.0 mm hollow fiber assumes about 0.3 mm to about 0.5 mm of meat growth around the outer diameter. An end diameter of approximately 1.1 mm can result in meat with about 85 hollow fibers/cm2.

In another embodiment, it is contemplated that the fibers have varying degrees or amounts of space between fibers. For example, having rows of fibers at a higher density interspersed between fibers at a lower density may be used to produce changes in the texture of the final structured clean meat product, such as is common in natural fish meat. Further still, it is contemplated that fibers of varying diameters, porosities and wall thicknesses may be used in the same hollow fiber cartridge, again, to simulate the appearance, texture, handleability and mouth feel of natural meat.

In any configuration, the fibers are spaced such that the spacing between the fibers is of a distance that permits an adequate flow of media (and the nutrients, growth factors, etc., contained therein) to reach all of the cell mass. This, of course, will be related at least in part on flow rate of the media and porosity of the hollow fiber walls but is related in greater part on physical distance from the surface of the outer wall of the hollow fiber to the cells. In other words, media and nutrients will only travel or defuse a limited distance through a cell mass. It is currently thought that the maximum for diffusion of oxygen and nutrients is 200 μm. Rouwkema, J., et al., (2009) Supply of Nutrients to Cells in Engineered Tissues, Biotechnology and Genetic Engineering Reviews, 26:1, 163-178. Thus, spacing between fibers should be about 400 μm from the outer wall of one fiber to the outer wall of a neighboring fiber. In culture conditions where media flows both through the hollow fibers and through the spacing between the hollow fibers the spacing can be greater. For example, spacing could be 800 μm from the outer wall of one fiber to the outer wall of a neighboring fiber. These figures are if the culture process relies on diffusion alone. However, use of a pump (for example) will create a flow of media from the hollow fibers, through the cell culture space between the hollow fibers and to the housing exits (rather than relying on diffusion alone) allowing the fibers to be spaced further apart. For example, in some embodiments it is contemplated the maximum distance between fibers is from about 0.05 mm (50 μm) to about 5.0 mm; about 0.1 mm to about 3.0 mm; about 0.1 mm to about 2.0 mm; about 0.1 mm to about 1.0 mm or about 0.2 mm to about 0.5 mm or any distance between the stated values. While it is a preferred embodiment that media flows from the center of the hollow fibers through the culture to the housing exits, it is also contemplated that the media flow can be in the reverse direction or can be alternated from one direction to the other, as desired. Alternating the direction of the media flow is believed to assist in ensuring all cells have an adequate media supply.

It is an embodiment of the present invention that a degree of randomness will be inherent in the distancing of the hollow fibers of the present invention. The figures given in the previous paragraph are average fiber-to-fiber distances for a given assembly. In an embodiment of the present invention, spacers and/or assembly techniques may be used to ensure, normalize or control the distances between the fibers. See, for example, Han G, Wang P, Chung TS., Highly robust thin-film composite pressure retarded osmosis (PRO) hollow fiber membranes with high power densities for renewable salinity-gradient energy generation, Environ Sci Technol. 2013 Jul. 16; 47 (14): 8070-7. Epub 2013 Jun. 28 or Chun Feng Wana, Bofan Li a, Tianshi Yang a, Tai-Shung Chung, Design and fabrication of inner-selective thin-film composite (TFC) hollow fiber modules for pressure retarded osmosis (PRO), Separation and Purification Technology, 172:32-42, 2017.

Once the cell density becomes too dense or the thickness of the cell mass becomes too thick, the ability of the media to reach the cells furthest away from the hollow fiber becomes difficult. A lack of media to these cells may result in dead cells in the reactor and/or dead spaces where cells cannot grow. The corollary is that the media needs to flow through the hollow fiber cartridge to the housing exits. That is, a flow of media needs to be maintained at least until confluency is reached and the structured clean meat product is harvested. One of skill in the art, based on the teachings of this specification, will be able to calculate the correct spacing of and porosity of the fibers of the present invention for a given desired structured clean meat product.

The hollow fibers of the present invention can be arranged and secured in what is referred to herein as a “hollow fiber cartridge.” In one embodiment, it is contemplated that the hollow fiber cartridge is made by having the ends of the hollow fibers are secured in an end piece in the desired arrangement. For example, each fiber has a first end and a second end. Each end is secured in an end piece, that is, a first and a second end piece. An end piece can be, for example, a resin or plastic that is known in the art to be inert and non-toxic to cells. At least one of the first or second ends of the hollow fibers is positioned in the end piece such that the interior lumen of the hollow fiber is in fluid communication with the exterior environment. Thus, with this positioning of the hollow fibers in the end piece, media can be caused to flow from the exterior environment of the hollow fiber (i.e., outside of the hollow fiber but inside of, for example, a sterile bioreactor) into the inner lumen of the hollow fiber.

One of skill in the art understands how to assemble hollow fibers into a module or cartridge. These techniques are applicable to the hollow fibers of the present invention. In brief, after spinning, the hollow fibers are cut to length and the ends of the fibers encased (i.e., potted) in a resin that will flow around the fiber ends and solidify. Sometimes, the section of the fibers may be encased in a substance (e.g., Plaster of Paris or other easily removable material known to one of skill in the art) to close the pores of the fibers so that the “potting solution,” i.e., the liquid resin, does not enter or plug the pores in the fibers. See, for example, Vandekar, V. D., Manufacturing of Hollow Fiber Membrane, Int'l J Sci & Res, 2015, 4:9, pp. 1990-1994, and references cited therein. In the present invention, one or both of the ends of the “potted” bundle are trimmed or cut to expose the open ends of the fibers to permit the flow of media once the bundle is inserted into a housing for use in the production of the structured clean meat of the present invention.

Further still, it is contemplated in some embodiments that the hollow fiber cartridge of the present invention has securing devices to maintain a desired distance between the first and second end piece. This may be necessary or preferred, for example, for easier insertion of the hollow fiber cartridge of the present invention into, e.g., a bioreactor housing.

Thus, it is contemplated that in one embodiment the hollow fiber cartridge of the present invention contains a plethora of hollow fibers arranged in a desired arrangement. The hollow fibers of the present invention have a first end and a second end. The arrangement is maintained by securing the first end and the second end of the hollow fibers in a first and a second end piece. The hollow fibers, once secured as describe, are then positioned parallel, substantially parallel or essentially parallel to each other. Further, the first and second end pieces are positioned parallel, substantially parallel or essentially parallel to each other. Further still, the hollow fibers of the hollow fiber cartridge of the present invention are positioned perpendicular, substantially perpendicular or essentially perpendicular to the end pieces of the hollow fiber cartridge of the present invention. The diameter and length of the hollow fiber cartridge will depend on the desired structured clean meat product being produced and bioreactor configurations.

In an embodiment of the present invention, it is contemplated that the hollow fibers of the hollow fiber cartridge of the present invention are at an average density of about 40-about 120 per cm², at an average density of about 60-about 100 per cm², at an average density of about 70-about 90 per cm² or any value between the values given above but not specifically iterated.

In an embodiment of the present invention, it is contemplated that the hollow fibers in the hollow fiber cartridge of the present invention have a void space between the hollow fibers prior to the addition of cells and, the void space between the hollow fibers is about 25%—about 75% of the total area of the hollow fiber cartridge or about 40%—about 60% of the total area of the hollow fiber cartridge or any value between the values given above but not specifically iterated.

In an embodiment of the present invention, it is contemplated that the hollow fiber cartridge of the present invention is designed to be removably inserted into a housing. That is, the cartridge can be inserted into the housing at the beginning of a production run and removed, i.e., harvested, at the end of the production run for any further desired processing of the structured clean meat product of the present invention. After harvesting of the structured clean meat product, a new hollow fiber cartridge of the present invention may be inserted into the housing and the process repeated. In this regard, the housing for the hollow fiber cartridge of the present invention is part of a bioreactor or bioreactor system.

Reactor configuration. The present invention is not limited to any particular reactor configuration or reactor system configuration so long as adequate media flow can be maintained through the culture and waste products removed. Hollow fiber reactors are typically tubular in shape although they can be oval, flat (sheet-like), rectangular or any other shape. In a preferred embodiment, the reactor comprises an insertable/removable insert that comprises the hollow fibers of the present invention. After confluent cell growth (as defined herein) is reached the insert can be removed and product finalized by removal of the insert ends and any further desired processing. Further processing may take the form of, for example, slicing, surface texturing, adding flavors, etc. Alternatively, further meat enhancement can take place before the harvest and disassembly of the device. For example, the media can be flushed out of the hollow fiber device and then the additives would be pumped directly into or around the fibers.

Non-limiting examples of suitable reactor systems. The most suitable type of reactor system is the feed batch system although it is contemplated that any available reactor will be suitable for use with the hollow fibers and hollow fiber cartridge of the present invention. For example, the MOBIUS® system (MilliporeSigma, Bedford, MA) is an example of a commercial system that can easily be converted to use with the present invention. The bioreactor in which the structured clean meat product is produced (i.e., the reactor comprising the hollow fibers of the present invention) may be seeded with cells grown in another bioreactor. The bioreactor that is seeding the hollow fiber device (a reactor suitable for cell growth (proliferation) and cell expansion) can be an existing commercial reactor, for example, a stirred tank or wave-type reactor. The proliferation/expansion bioreactor is contemplated to be, for example, a stirred tank or wave-type reactor (as are known to one of ordinary skill in the art) and to be a suspension, agglomerated biomass, microcarrier culture, or other suitable reactor known to one of ordinary skill in the art. It is contemplated that the production bioreactor (i.e., the reactor comprising the hollow fibers of the present invention) may be, for example, single use, multi-use, semi-continuous or continuous. The present invention further contemplates a manifold of multiple reactors comprising the hollow fiber of the present invention.

Thus, it is contemplated that an exemplary reactor system of the present invention comprises one of more hollow fiber cartridges of the present invention, a housing sized to hold said hollow fiber cartridge; a medium source fluidly connected to one or more inlets in said housing; one or more medium outlets in said housing; and one or more pumps to supply the medium to and/or remove waste medium from said hollow fiber cartridge through said medium inlet(s) and/or outlet(s). Further still, the inlets are fluidly connected to the interior of the hollow fibers. Yet further still, the hollow fiber bioreactor may comprise an automated controller or automatically controlled system.

The present invention also contemplates a process for producing a meat product, comprising; seeding a void space between the hollow fibers in a hollow fiber reactor of the present invention with one or more of myocytes, myocyte-like cells or engineered cells expressing one or more myocyte-like characteristics at a density of, for example, 100,000 cells to 100,000,000 (10⁵-10⁸) (Radisic, et al., Biotechnol Bioeng, 2003 May 20: 82 (4): 403-414.) and culturing the cells until achieving about 80%—about 99% confluency, 85%—about 99% confluency, about 90%—about 99% confluency, about 95%—about 99% confluency, about 98%—about 99% confluency or about 100% confluency (or any value in between the recited percent values), removing said first holding device and said second holding device from the first ends and second ends, respectively, of said hollow fibers.

After seeding, the hollow fiber cartridge has media supplied to the cells through one or both of the first end and second end of the hollow fibers into the interior of the hollow fibers, through the wall of the hollow fibers into the void space between the hollow fibers where said cells are seeded and through one or more of said outlets in said housing. In another embodiment, it is contemplated that media can also flow between fibers from both the inlet(s) and outlet(s) of device. For example, one fluid path is through fiber wall and the second fluid path is around the fibers. It is contemplated that the device may have multiple inlets and outlets. After the cells achieve confluency, flushing out any residual media and waste products and infusing the interior of the hollow fibers and/or any remaining void space between the cells with one or more of fats, flavors, colors, salts and preservatives.

Fats suitable for addition to the structured clean meat product of the present invention include, but are not limited to: saturated, monounsaturated, polyunsaturated fats such as corn oil, canola oil, sunflower oil, and safflower oil, olive oil, peanut oil, soy bean, flax seed oil, sesame oil, canola oil, avocado oil, seed oils, nut oil, safflower and sunflower oils, palm oil, coconut oil, Omega-3, fish oil(s), lard, butter, processed animal fat, adipose tissue, or cellular agriculture derived fat, or combinations thereof. Synthetic fats such as oleoresin may also be used. In fact, any fat recognized by the Food and Drug Administration (FDA) is suitable for use in the present invention and contemplated for use in the structured clean meat product of the present invention. On the FDAs food additive list, natural substances and extractives (NAT), Nutrient (NUTR), Essential oil and/or oleoresin (solvent free) (ESO).

Flavors suitable for use in the structured clean meat product of the present invention include, but are not limited to, any flavor documented on the FDA's food additive list. These may be documented as natural flavoring agents (FLAV), essential oils and/or oleoresin (solvent fee) (ESO), enzymes (ENZ), natural substances and extractives (NAT), non-nutritive sweetener (NNS), nutritive sweetener (NUTRS), spices, other natural seasonings & flavorings (SP), synthetic flavor (SY/FL), fumigant (FUM), artificial sweeteners including aspartame, sucralose, saccharin and acesulfame potassium and yeast extract, or combinations thereof, are contemplated for use in the structured clean meat product of the present invention.

Texture Enhancers suitable for use in the structured clean meat product of the present invention include, but are not limited to, pureed plant material, guar gum, cellulose, hemicellulose, lignin, beta glucans, soy, wheat, maize or rice isolates and beet fiber, pea fiber, bamboo fiber, plant derived fiber, plant derived gluten, carrageenan, xanthan gum, lecithin, pectin, agar, alginate, and other natural polysaccharides, grain husk, calcium citrate, calcium phosphates, calcium sulfate, magnesium sulfate and salts, or any combination thereof, are contemplated for use in the structured clean meat product of the present invention. These may be documented on the FDA's food additive list as solubilizing and dispersing agents (SDA), and natural substances and extractives (NAT).

Nutritional Additives suitable for use in the structured clean meat product of the present invention include, but are not limited to, vitamins, trace elements, bioactive compounds, endogenous antioxidants such as A, B-complex, C, D, E vitamins, zinc, thiamin, riboflavin, selenium, iron, niacin, potassium, phosphorus, omega-3, omega-6, fatty acids, magnesium, protein and protein extracts, amino acids salt, creatine, taurine, carnitine, carnosine, ubiquinone, glutathione, choline, glutathione, lipoic acid, spermine, anserine, linoleic acid, pantothenic acid, cholesterol, Retinol, folic acid, dietary fiber, amino acids, and combinations thereof, are contemplated for use in the structured clean meat product of the present invention. Any food additive or additives that are generally recognized as safe (GRAS) or approved by the FDA are contemplated for use in the structured clean meat product of the present invention and incorporated herein. See, for example: www.fda.gov/food/food-additives-petitions/food-additive-status-list.

Any food coloring or colorings, natural or artificial, that are Generally Recognized As Safe (GRAS) or approved by the FDA are contemplated for use in the structured clean meat product of the present invention. See, for example: www.fda.gov/industry/color-additive-inventories/color-additive-status-list.

Prophetic cell types. The hollow fibers of the present invention are designed to be used to grow specific cell types suitable for the production of in vitro or lab grown meat and meat products, i.e., the structured clean meat of the present invention. Therefore, while many different types of cells can grow on the hollow fibers (and in the hollow fiber cartridges of the present invention, if desired), the fibers were developed to be used to grow muscle cells (i.e., myocytes), or cells with the characteristics of muscle cells or engineered to have the characteristics of muscle cells (collectively referred to herein as muscle cells or myocytes), to confluency and to mimic the natural structure of muscle (i.e., meat). Preferably, the muscle is skeletal muscle. That is, the hollow fibers of the present invention are designed by the inventors to be suitable to grow myocytes to obtain muscle fibers or myofibrils. Further, other types of cells may be grown on the hollow fibers of the present invention and in reactors comprising the hollow fibers of the present invention. These cells may be grown independently or in combination with muscle cells. For example, adipocytes or cells having the characteristics of adipocytes or engineered to have the characteristics of adipocytes (collectively referred to herein as adipocytes) may be cultured with the muscle cells to achieve an end product resembling natural muscle or meat. The hollow fibers of the present invention are also suitable for including other cells to be co-cultured with the muscle cells of the present invention, for example, fibroblasts, cells having the characteristics of fibroblasts or cells engineered to have the characteristics of fibroblasts.

With specific regards to a co-culture of muscle cells and adipocytes, the ratio of muscle cells to adipocytes may be 99:1, 95:5, 92:8, 90:10, 88:12, 85:15 82:18, 80:20, 75:25 or any ratio from 100:0 to 75:25, inclusive.

The cells that are suitable for use with the present invention may be obtained from or derived from any animal from which food is now obtained. Prominent examples are bovine, porcine, ovine, piscine (e.g., fish such as tuna, salmon, cod, haddock, shark, etc.), shellfish, avian (e.g., chicken, turkey, duck, etc.). More exotic sources of cells may also be used, such as from animals that are traditionally hunted rather than farmed (e.g., deer, elk, moose, bear, rabbit, quail, wild turkey, etc.) or combinations thereof.

Cells used in the present invention may be derived by any manner suitable for the generation of differentiated cells having the characteristics desired. For example, any procedure suitable for deriving cells with differentiated myocyte-like characteristics, adipocyte-like characteristics, etc. Such characteristics for myocytes include, for example, but not necessarily limited to, having an appearance of a long, tubular cell and with large complements of myosin and actin. Myocytes also have the ability to fuse with other myocytes to form myofibrils, the unit of muscle that helps to give muscle, i.e., meat, its distinctive texture. Such characteristics for adipocytes (also referred to in the art as lipocytes and fat cells) include, for example, but not necessarily limited to, having large lipid vacuoles that may take up as much as 90% or more of the volume of the cell. The hollow fibers of the present invention provide, at least in part, a replacement of the connective tissue (referred to as “fascia” in the art) typically found in skeletal muscle.

Cells useful in the present invention include, but are not limited to, cells that are derived from mesenchymal stem cells or induced pluripotent stem cells (iPSC). iPSCs are cells engineered to revert to their pluripotent state from which numerous cells types can be derived. In other words, iPSCs are pluripotent stem cells that can be generated directly from a somatic cell. The technology was first reported in 2006 (Takahashi K, Yamanaka S, 25 Aug. 2006, “Induction of pluripotent stem cells from mouse embryonic and adult fibroblast cultures by defined factors” Cell, 126 (4): 663-76), has advanced from that point on (see, for example: Li, et al., 30 Apr. 2014, “Generation of pluripotent stem cells via protein transduction” Int. J. Dev. Biol., 58:21-27), includes the generation of muscle cells (see, for example: Rao, et al., 9 Jan. 2018, “Engineering human pluripotent stem cells into a functional skeletal muscle tissue” Nat Commun., 9 (1): 1-12) and is well known to one of ordinary skill in the art.

Unless defined otherwise, all technical and scientific terms used herein have the same meaning as commonly understood by one of ordinary skill in the art to which this invention pertains.

When introducing elements of the present disclosure or the preferred embodiments(s) thereof, the articles “a,” “an,” “the” and “said” are intended to mean that there are one or more of the elements. The terms “comprising,” “including” and “having” are intended to be inclusive and mean that there may be additional elements other than the listed elements.

The transitional phrases “comprising,” “consisting essentially of” and “consisting of” have the meanings as given in MPEP 2111.03(Manual of Patent Examining Procedure, 9^{th} Ed., Revision 10.2019; United States Patent and Trademark Office). Any claims using the transitional phrase “consisting essentially of” will be understood as reciting only essential elements of the invention and any other elements recited in claims dependent therefrom are understood to be non-essential to the invention recited in the claim from which they depend.

All ranges recited herein include all values within the cited range including all whole, fractional and decimal numbers, inclusive.

### Exemplification

### General Materials and Methods

All reagents were commercially available and used without further purification unless otherwise stated. Zein, sodium hydroxide, urea, hydroxypropyl cellulose, k-carrageenan, sodium acetate, tripolyphosphate (TPP), (hydrocholeretic acid 37%, Antibiotic Antimycotic Solution (100×) and wheat gluten, fibronectin from bovine serum were purchased from MilliporeSigma (Burlington MA). Bovine collagen was purchased from Corning (Corning, NY); soy protein isolate (SPI) from BulkSupplements (Henderson, NV); chitosan (from mushrooms) was purchased from Modernist Panty (Elliot, ME, USA); pea and peanut butter protein isolates were purchased from NorCal Organic (Crescent City, CA); mung bean, fava bean, and chickpea protein isolates were purchased from Green Boy (Redondo Beach, CA); agarose was purchased from Hispanagar (Burgos, Spain); brown rise protein isolate was purchased from Zen Principle (Incline Village, NV); and Sodium alginate and MooGloo™ RM transglutaminase were purchased from Modernist Pantry (Eliot, ME).

### Cell-Media Stability Tests:

Membranes were cut into 1×3.5 inches square samples and incubated in cell media containing Antibiotic Antimycotic Solution (2×) (known to one of skill in the art) at 37° C. up to 21 or 30 days depending on the experiments. For each membrane type, three samples were mechanical tested during the incubation at each time point.

### Viscosity:

Viscosity measurements of the prepared dope solutions were taken on a Brookfield (Middleboro, MA) Viscometer DV-II+ Pro using the S64 spindle.

### Mechanical Testing:

Membrane tensile tests were performed on 1×3.5 or 0.5×3.5 inches square sample using a Zwick Roell (Kennesaw, GA) a TestControl II device and the data were analyzed using Zwick Roell testXpert II V3.71 software.

### Freeze-Drying:

Samples were frozen in water under liquid nitrogen over 1 hour inside a scintillation vial. Afterwards, the frozen samples were dried using a Labconco (Kansas City, MO) freeze-drier 2.5 L −80° C.

### Scanning Electron Microscopy:

Samples are mounted on the stub, coated with 3 nm of iridium and imaged either using a ThermoScientific (Waltham, MA) Quanta 200F or a JOEL (Peabody, MA) JCM 6000 scanning electron microscope (Wrn|r/Mdsdq.

### Statistical Analysis:

Error bars are calculated as standard error of the mean.

### Rheology:

Rheological analyses on the formulated dope and membranes were carried out on a TA Ares rheometer (New Castle, DE) using conical fixtures.

### Example 1—Method of Producing Edible Hollow Fibers

Refer to FIGS. 1 & 2 for schematic representations of exemplary production processes for producing the membranes of the present invention.

### 1. Creating the Dope Solution

a. Making the dope solution requires a multistep mixing process

i. First the protein solution was made. This required the dissolution of 14% plant protein concentrate by weight in weak alkaline buffer solution. The mix was homogenized at 20,000 rpm for several minutes. Specifically, micronized plant protein powder was used.

ii. The second solution contains the carrier polymer comprising 2% alginate, 2% hydroxypropyl cellulose dissolved in the same buffer as the protein mix. This was dissolved by hybridizer at 35° C. for 48 hours.

iii. In a 1:1 ratio the protein solution and carrier polymer solutions were mixed. The mixing was completed with an overhead stirring apparatus followed by 12 hours in a hybridizer at 35° C.

iv. The final mix has a resulting concentration of 2% polysaccharide and 7% plant protein and is referred to as the dope solution.

b. Making the bore solution is completed by dissolving 15 g/l calcium chloride in reverse osmosis (RO) water with 0-1 g/l transglutaminase.

### 2. Drawing and Solidification

a. Using a pressurized vessel and gear pump, the dope solution is pushed through a coaxial orifice. There was a specific distance between the spinneret and the bath, which may be adjusted based on the dope solutions rheological properties.

b. The solidification bath (also referred to herein as the formation bath) is also 15 g/l calcium chloride and locked in the 3D structure of the fiber by ionically crosslinking the alginate.

### 3. Crosslinking Step

a. For this application, ionic crosslinking of the alginate may not serve sufficient for the dissociation of the divalent bond by the monovalent bond made by the sodium salt in the cell culture media. Crosslinking beyond the enzymatic transglutaminase crosslink and the alginate-calcium crosslink was desired.

b. The fiber was then exposed to heat close to 100° C. to thermally crosslink the proteins within the fiber. Proof of concept has been demonstrated via autoclave at 121° C. for 60 minutes.

c. Alternatively, or in addition, the fiber was exposed to electron beam or gamma irradiation at approximately 50 kGy (kilogray) to physically crosslink the cellulose portion of the mix, i.e., to crosslink the proteins. The final dosage can be from approximately 5 kGy to approximately 100 kGy depending on the residence time of the material passing through the electron beam and the grade of the materials, as can be determined by one of skill in the art utilizing the teachings of this specification.

### 4. Coating Step

a. The fiber was continuously passed through a plasma chamber then dipped into a solution of 15% glycol/sorbitol (1:1) mix in water (depending on the use, the ratio of glycol/sorbitol may range from 1:14 to 14:1). This step was designed to minimize the collapse of the porous structure of the hollow fiber via plasticizer.

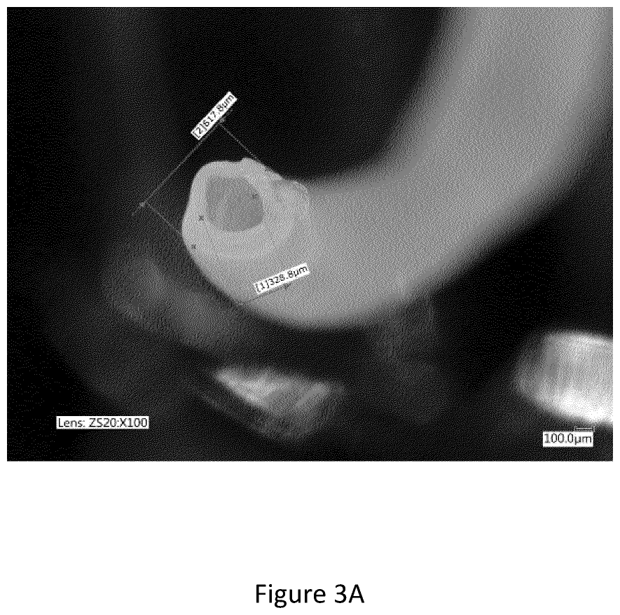

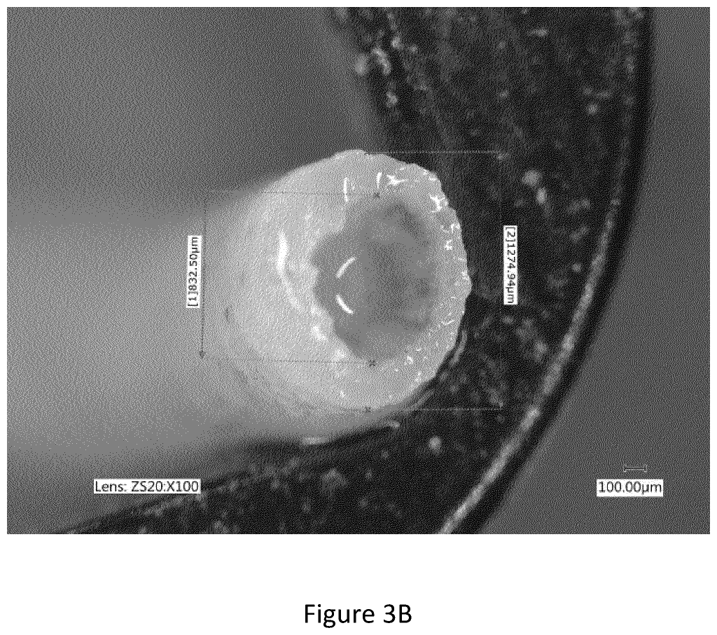

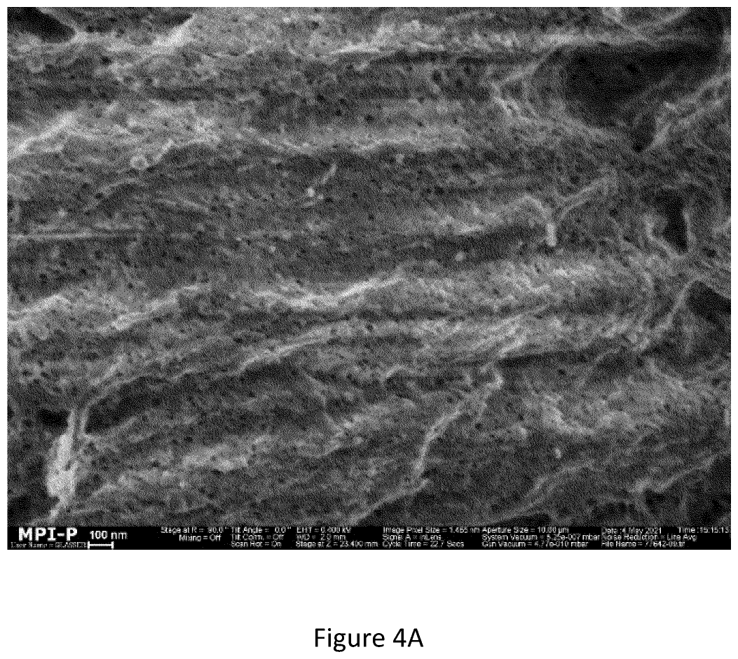

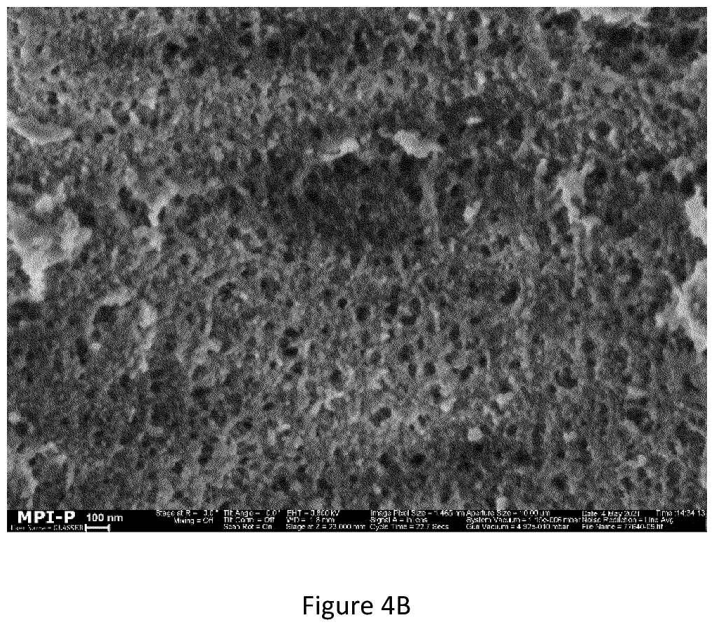

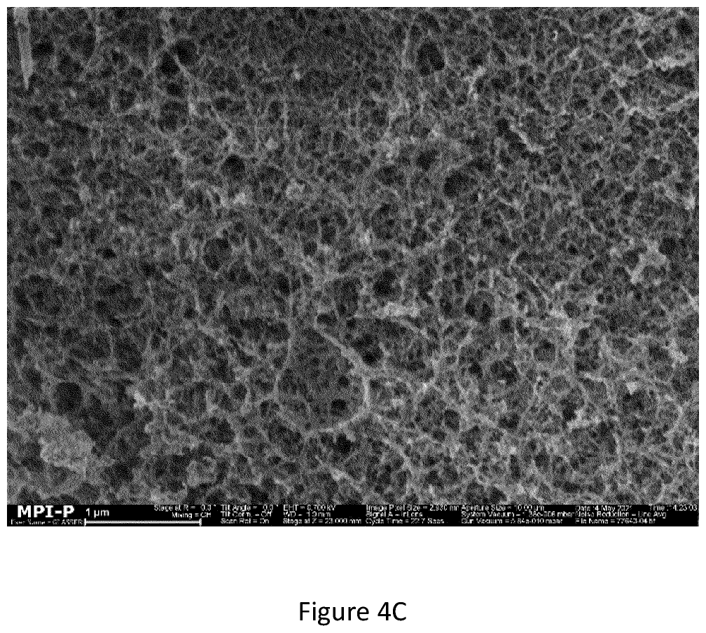

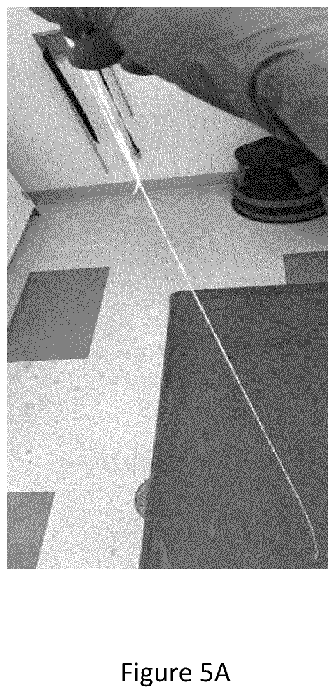

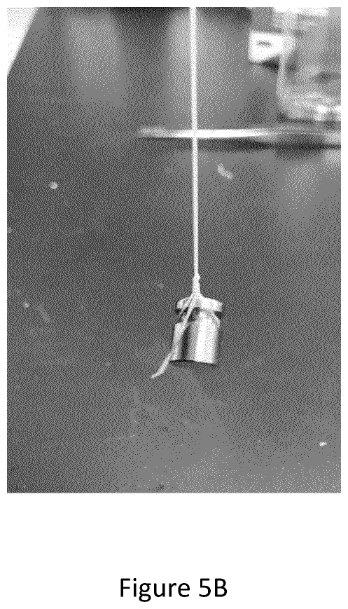

FIGS. 3A & B show micrographs of hollow fiber membranes made with the process (method) of Example 1. FIGS. 4A-C show scanning electron micrographs of hollow fiber membranes made with the process of this example. FIG. 5A shows the length of one hollow fiber made with the process of this example. FIG. 5B provides a demonstration of tensile strength of one of the hollow fibers.

### Example 2—Prophetic Example of Fibers without Secondary Crosslinking Step

a. Hollow fiber dope solution is created as defined above in Example 1 is used. In this example, three conditions are targeted. All conditions form from the same dope solution. This dope solution is 1-part hydroxypropyl cellulose, 1-part alginate acid sodium salt (Sigma Aldrich, St. Louis, MO), and 7 parts pea protein isolate.

b. In the first condition the fibers are extruded directly into the 15 g/l Calcium chloride bath, instantly solidifying. After 10 minutes in the bath, the fibers are rinsed with MilliQ™ water (MilliporeSigma, Bedford, MA) water and then submerged in DMEM F12 media for 72 hours. Upon removing the fibers from the cell culture media, they are not handleable. The fibers can no longer support their own weight outside of the solution. The majority of the ionic crosslinked sites have dissociated.

c. In the second condition the fibers are extruded directly into the 15 g/l Calcium chloride bath, instantly solidifying. After 10 minutes in the bath, the fibers are rinsed with MilliQ™ water and then autoclaved at 121° C. for 30 minutes. Once cooled to room temperature the fibers are then submerged in DMEM/F12 (Dulbecco's Modified Eagle Medium/Nutrient Mixture F-12; ThermoFisher Scientific, Waltham, MA) media for 72 hours. Upon removing the fibers from the cell culture media, they have lost some degree of integrity. The fibers can be removed but can only self-support approximately 5 inches of itself. Most of the ionic crosslinked sites have dissociated, however the protein that has been thermally crosslinked is still responsible for increasing fiber integrity.

d. In the third condition the fibers are extruded directly into the 15 g/l Calcium chloride bath, instantly solidifying. After 10 minutes in the bath, the fibers are rinsed with MilliQ™ water and then exposed to a single pass at 50 kGy in benchtop electron beam modification equipment, submerged in DMEM/F12 media for 72 hours. Upon removing the fibers from the cell culture media, they maintain their integrity and can support their own weight. Though ionically crosslinked sites are susceptible to dissociation in the cell culture media, and there may be some chain scission of the backbone of both the alginate and cellulose, the physical crosslinking of the protein polymer network is resistant to dissociation in the media.

These examples show that the crosslinking of the protein with heat and/or irradiation results in enhanced integrity of the hollow fibers of the present invention making them suitable for use in, for example, cell culture apparatuses or filtration devices.

### Example 3—Dope Solution Preparation

### 1.1. Protein Solutions

### 1.1.2. Urea-Based Method:

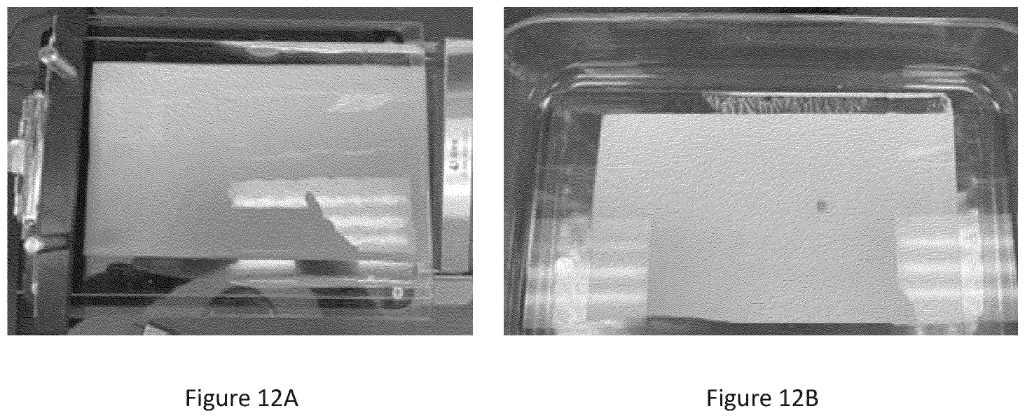

Zein: A zein solution (15% w/v) was prepared by adding 57 g of zein powder to 300 mL of MilliQ™ water at 0° C. and under mechanical stirring. After 30 minutes, 11.25 g of urea was added to the suspension followed by the addition of 83 mL of NaOH solution (0.4 N) (FIG. 12). Afterwards, the reaction was allowed to warm up to room temperature (23° C.) and stirred for 18 hours before further use.

Zein: A zein solution (19% w/v) was prepared by adding 72 g of zein powder to 300 mL of MilliQ™ water at 0° C. and under mechanical stirring. After 30 minutes, 14.30 g of urea was added to the suspension followed by the addition of 83 mL of NaOH solution (0.6 N) (FIG. 12). Afterwards, the reaction was allowed to warm up to room temperature (23° C.) and stirred for 18 hours before further use.

Zein-Hydroxypropyl cellulose blend: A 0.5% w/v hydroxypropyl cellulose (HPC) solution (0.5% w/v) was prepared by adding 1.75 g of HPC in MilliQ™ water and mixing by mechanical stirring over 18 h. Afterwards, the solution was cooled down to 0° C. using an ice bath, and Zein (72 g) were added to it. The suspension was allowed to stir at 0° C. for an additional 20 minutes before adding 14.30 g of urea and 83 mL of NaOH solution (0.6 N). The reaction was allowed to warm up to room temperature (23° C.) and stirred for an additional 18 hours before further use.

Soy protein isolate: A soy protein isolate (SPI) solution (20% w/v) was prepared by adding 76 g of SPI powder to 300 mL of MilliQ™ water under mechanical stirring. After 30 minutes, 11.25 g of urea was added to the suspension followed by the addition of 83 mL of NaOH solution (0.4 N). Afterwards, the reaction was allowed to stir for 18 hours before further use.

Pea protein isolate: A soy protein isolate (SPI) solution (20% w/v) was prepared by adding 76 g of PPI powder to 300 mL of MilliQ™ water under mechanical stirring. After 30 minutes, 11.25 g of urea was added to the suspension followed by the addition of 83 mL of NaOH solution (0.4 N). Afterwards, the reaction was allowed to stir for 18 hours before further use.

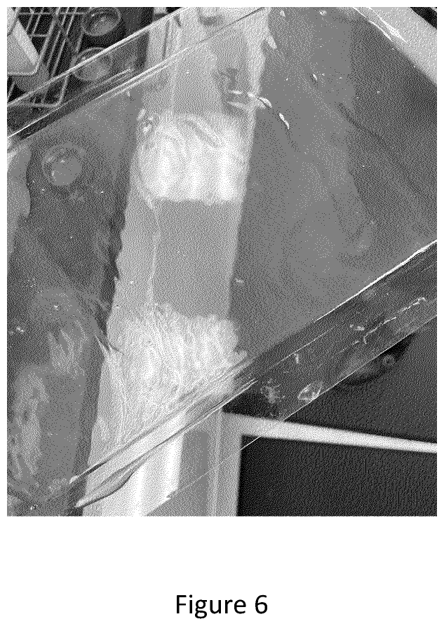

Mung Bean: A Mung Bean solution (15% w/v) was prepared by adding 57 g of PPI powder to 300 ml of MilliQ™ water under mechanical stirring. After 30 minutes, 11.25 g of urea was added to the suspension followed by the addition of 83 mL of NaOH solution (0.4 N). Afterwards, the reaction was allowed to stir for 18 hours before further use. See, FIG. 6.

Wheat gluten: A gluten solution (15% w/v) was prepared by adding 56 g of gluten powder to 300 mL of MilliQ™ water under mechanical stirring. After 30 minutes, 11.25 g of urea was added to the suspension followed by the addition of 83 mL of NaOH solution (0.4 N). Afterwards, the reaction was allowed to stir for 16 hours before further use.

### 1.1.3. Hybridizer-Based Method:

### Mung Bean Alginate Blend:

Mung bean protein isolate (Green Boy) and alginate (] Modernist Pantry) blends are formulated by weighing out 45 grams of mung bean protein isolate in 252 grams of water and homogenizing it at 25000 rpms for 5 minutes. From there 3 ml of 10N NaOH (and an optional 6 g of urea) is added and it is homogenized for 5 more minutes. From there, the gel-solution is placed into a homogenizer at 40° C. overnight.

### 1.2. Alginate-Protein Blend Solutions

### Alternative Mung Bean and Alginate Dope Formulation:

Therefore, ranging studies have been conducted finding multiple possible formulations of protein isolate and alginate. One exemplary formulation and mixing method is expressed in weight: 0.2% Alginate, 15% mung bean protein isolate, 1% 10N NaOH, 2% urea (optional), 81.8% MilliQ™ water.

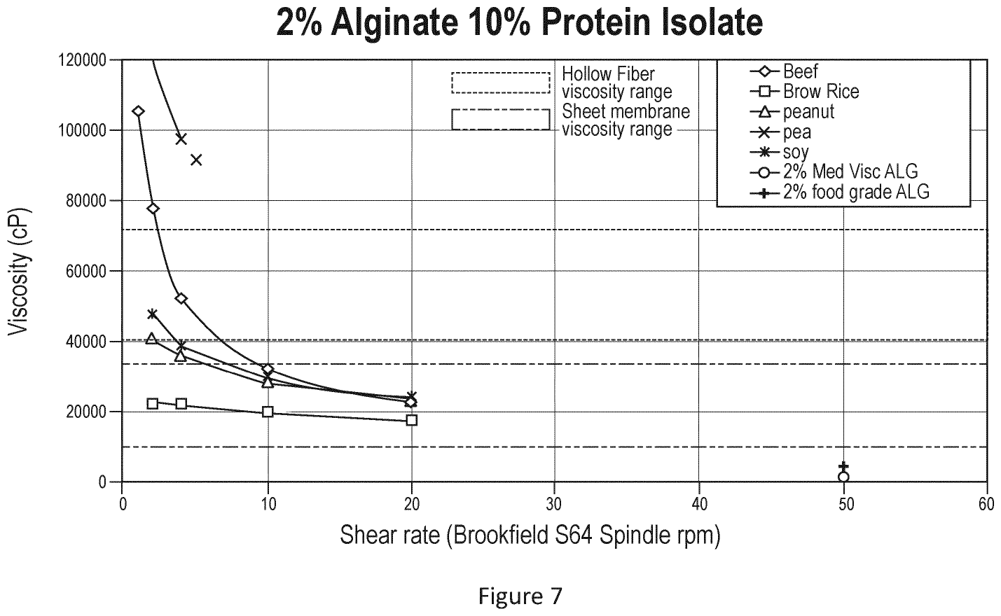

The first step is to wet out (i.e., suspend) and disperse the protein isolate in solution. The protein isolate is weighed out and the MilliQ™ water is added. A high shear mixer such as a homogenizer (IKA, Staufen, Germany) is set to 25,000 rpms for 5-10 minutes, or until the slurry returns to fluid like behavior. Once disperse, the NaOH (and if desired-urea) is added and to the protein and water the solution is then homogenized for an additional 5 minutes until a viscous gel is formed. From there, an overhead mixer fit with a propeller is set to 100-500 rpms to stir the dissolved protein. The Alginate is slowly added to the mixing solution for over the course of 15 minutes. Once the alginate is homogenously dispersed throughout the mix and partially dissolved, the solution is put into a jar, capped and placed Into a hybridizer for 24 hours. See, FIG. 7.

### i.1.2.1. Urea-Based Method

Zein-Alginate: Zein-alginate blends of different biopolymers ratios were prepared by mixing under mechanical stirring, for 20 minutes, zein solutions (15% w/v), prepared according to the urea-method, with pre-made alginate water solutions of varying concentrations (2% w/v, 4% w/v, 6% w/v and 8% w/v).

SPI-Alginate: SPI-alginate blends of different biopolymers ratios were prepared by mixing under mechanical stirring, for 20 minutes, SPI solutions (20% w/v), prepared according to the urea-method, with pre-made alginate water solutions of varying concentrations (2% w/v, 4% w/v, 6% w/v and 8% w/v).

PPI-Alginate: SPI-alginate blends of different biopolymers ratios were prepared by mixing under mechanical stirring, for 20 minutes, PPI solutions (20% w/v), prepared according to the urea-method, with pre-made alginate water solutions of varying concentrations (2% w/v, 4% w/v, 6% w/v and 8% w/v).

Mung bean-Alginate: Mung bean-alginate blends of different biopolymers ratios were prepared by mixing under mechanical stirring, for 20 minutes, Mung bean solutions (15% w/v), prepared according to the urea-method, with pre-made alginate water solutions of varying concentrations (2% w/v, 4% w/v, 6% w/v and 8% w/v).

Gluten-Alginate: Gluten-alginate blends of different biopolymers ratios were prepared by mixing under mechanical stirring, for 1 hour, gluten solutions (15% w/v), prepared according to the urea-method, with pre-made alginate water solutions of varying concentrations (2% w/v, 4% w/v, 6% w/v and 8% w/v).

### 1.3. Protein-Agarose Blend Solutions

### 1.3.1. Urea-Based Method

Zein-Agarose: Zein-agarose blends of different biopolymers ratios were prepared by mixing zein solutions (15% w/v), prepared according to the urea-method, with pre-made agarose water solutions of varying concentrations (1% w/v, 2% w/v and 4% w/v). The agarose solutions were prepared by adding the respective amounts of agarose in 300 ml of MilliQ™ water at 60° C. and letting them stir for 2 hours until dissolution was complete. To obtain homogeneous blends and avoid solidification of agarose before casting, the freshly prepared agarose solutions were added to the pre-heated zein solutions at 40° C. and allowed to stir for 20 minutes. The solutions were kept at 40° C. before casting.

Although explored with Zein-Agarose, the formulation results and method is expected to have similar results with other plant-based proteins. Formulating an agarose and Corn Protein (zein) is not a trivial task as the two polymers do not utilize common solvents or dissolution temperatures. A combination of stabilizing the zein above 40° C. as well as lowering the required percent of ethanol that is required to levels below about 20%, is achieved simultaneously. Using Minitab (State College, Pennsylvania) design of experiments for formulations, a solvent system with 0.04 N sodium hydroxide, urea, and ethanol are explored by percent w/v.

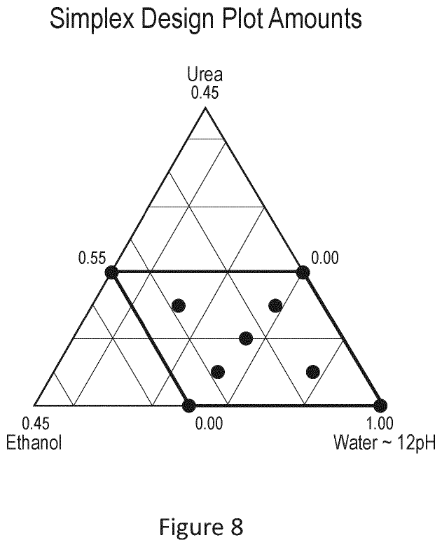

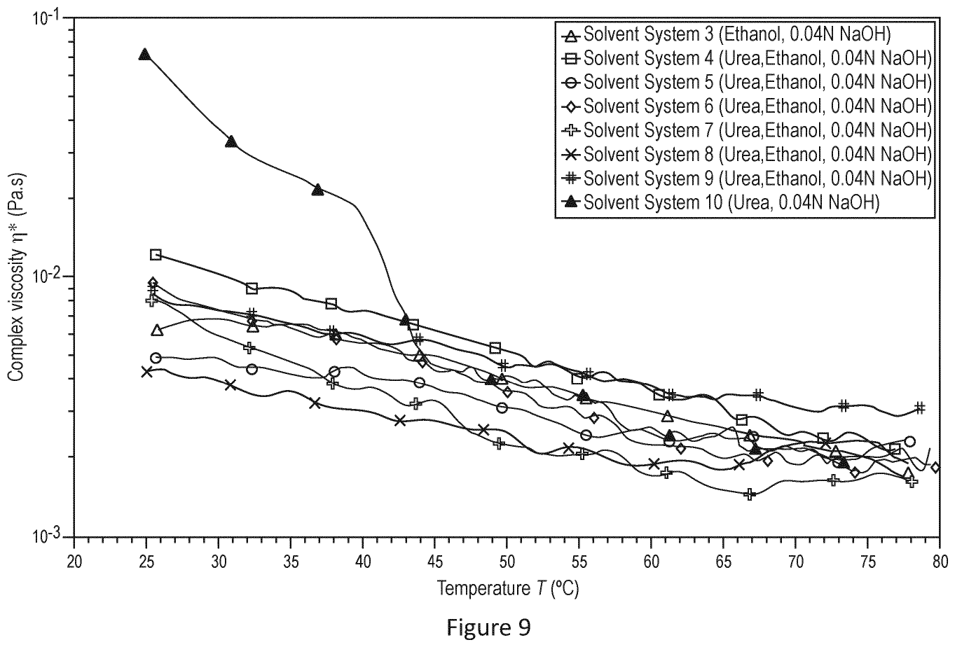

It was found that the combination of ethanol, urea, and 0.04N NaOH was able to dissolve zein. Surprisingly, zein was able to dissolve at ethanol contents at 10% in the presence of 0.04N NaOH and urea. A simplex design plot is shown in FIG. 8. However, zein without ethanol was not stable at temperatures below about 40° C. This observation is supported with the temperature-sweep rheology data. See, FIG. 9.

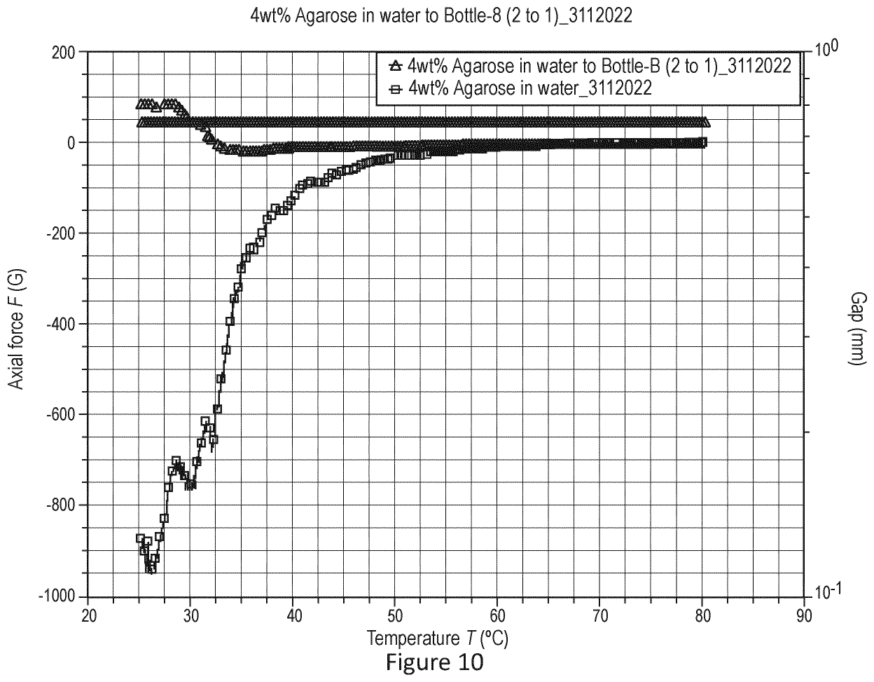

Furthermore, this solvent system composed of about 5% urea, 19% ethanol, and 76% 0.04 N NaOH was found to reduce the gelation properties of agarose. See, FIG. 10.

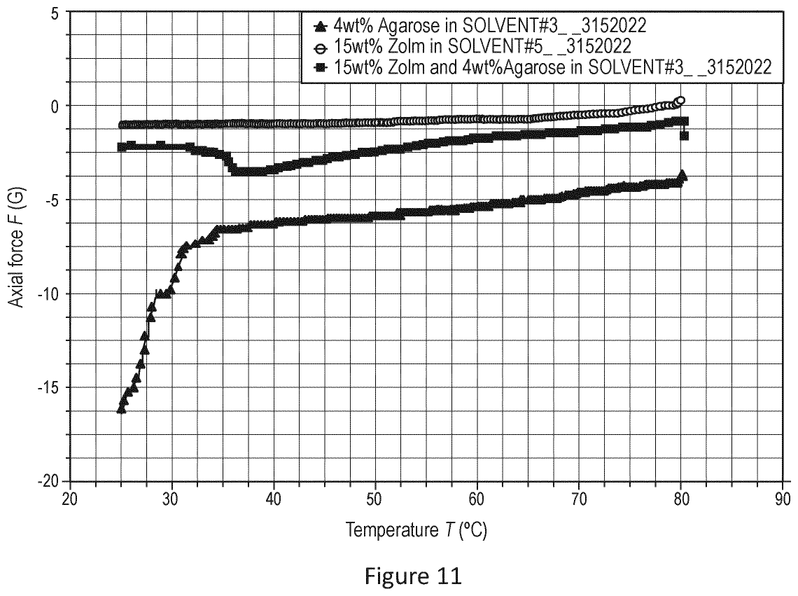

Furthermore, when we mix agarose and zein together in this solvent system we see the rheological properties that indicate feasibility of mixing both polymers together within one system See, FIG. 11.

Mung bean-agarose: Mung bean-agarose blends of different biopolymers ratios were prepared by mixing mung bean solutions (15% w/v), prepared according to the urea-method, and pre-made agarose water solutions of varying concentrations (1% w/v, 2% w/v and 4% w/v). The agarose solutions were prepared by adding the respective amounts of agarose in 300 mL of MilliQ™ water at 60° C. and letting them stir for 2 hours until complete dissolutions. To obtain homogeneous blends and avoid solidification of agarose before casting, the freshly prepared agarose solutions were added to the pre-heated mung bean solutions at 40° C. and allowed to stir for 20 minutes. The solutions were kept at 40° C. before casting.

Gluten-Agarose: Gluten-agarose blends of different biopolymers ratios were prepared by mixing gluten solutions (15% w/v), prepared according to the urea-method, and pre-made agarose water solutions of varying concentrations (1% w/v, 2% w/v and 4% w/v). The agarose solutions were prepared by adding the respective amounts of agarose in 300 ml of MilliQ™ water at 60° C. and letting them stir for 2 hours until complete dissolutions. To obtain homogeneous blends and avoid solidification of agarose before casting, the freshly prepared agarose solutions were added to pre-heated zein solutions at 40° C. and allowed to stir for 20 minutes. The solutions were kept at 40° C. before casting.

### 1.4 Plant-Based Chitosan

Mushroom-based Chitosan: was purchase from Modernist Pantry. Varying concentrations of the chitosan (5% w/v and 7% w/v) were dissolved in 5% Acetic Acid via 35° C. hybridizer overnight. A formation bath containing 10 g/L triphenyl phosphate was used for the solidification/crosslinking. Chitosan membranes were left to crosslink overnight before handling.

### 1.5 K-Carrageenan

K-carrageenan: K-carrageenan was heated to 90° C. in MilliQ™ water at varying concentrations (2% w/v, 4% w/v and 10% w/v). at the elevated temperature the solution was cast onto preheated plates and submerged into a formation bath containing 15 g/L of calcium chloride. In another scenario, K-Carrageenan was heated with the calcium chloride in the solution. Upon cooling, the solution solidified into the membrane.

b. Membrane Preparation/Formation

Membranes were casted either using an automatic film caster (BYK Drive 6 film caster, Leominster, MA) equipped with a 524 micron-gap bar or a hand-caster with a gap of 600 micron. In both cases, 40 mL of dope solution for each membrane was used and led to a membrane dimension of about 25×15 cm² area. Depending on the membrane formulation, different coagulation conditions were applied.

For hollow fibers, the dope solution was extruded though co-axial needles purchased from Ramé-hart instrument, Co. (Succasunna, NJ). Alternatively, a custom-made lab-scale hollow fiber spinning machine was used-allowing for the processing of much higher viscosities (up to 100,000 centipoise: cP).

### 2.1. Protein Membranes

Whether obtained through the urea or the hybridizer method, flat sheet protein membranes were casted into a sodium acetate buffer (0.2 M, pH 4.5) (see, FIGS. 12A & B) and allowed to equilibrate in the same buffer for 10 minutes up to 3 hours. Afterwards, membranes were washed with HEPES (0.1 M, pH 7.4) and stored in an ethanol/water solution 70/30% w/v.

Differently, zein membranes were stored in a HEPES buffer solution (0.1 M, pH 7.4) containing 2× of antibiotic antimycotic solution.

### 2.2. Protein-Alginate Blend Membranes

Whether obtained through the urea or the hybridizer method, flat sheet protein-alginate blend membranes were casted into a sodium acetate buffer (0.2 M, pH 4.5) containing CaCl₂) (15 g/L) and allowed to equilibrate in the same buffer for 10 minutes up to 3 hours. Afterwards, membranes were washed with HEPES (0.1 M, pH 7.4) containing CaCl₂) (15 g/L), and stored in an ethanol/water solution 70/30% w/v.

Using the doctoral blade technique (as is known to one of ordinary skill in the art), the alginate and mung protein mixture is coated onto a PTFE sheet. The sheet is then placed into an acetate buffer of 4.5 pH that contains 15 g/L calcium chloride. The shift from pH 11 to pH 4.5 caused the coagulation of the protein and the calcium chloride crosslinked the alginate. On the bench, the membrane sits in the buffer solution for 10 minutes. Once the membrane is formed and turned white (off white), the membrane is removed and put into a shaking 99.5% glycerin bath for 10 minutes.

### 2.3. Protein-Agarose Blend Membranes

Whether obtained through the urea or the hybridizer method, protein-agarose blend membranes were casted from hot solutions kept at 40° C. into a sodium acetate buffer (0.2 M, pH 4.5) and allowed to equilibrate in the same buffer for 10 minutes up to 3 hours. Afterwards, membranes were washed with HEPES (0.1 M, pH 7.4) containing CaCl₂) (15 g/L), and stored in an ethanol/water solution 70/30% w/v.

### 3. Membrane Cross-Linking:

3.1 Protein-Alginates Crosslinked with Transglutaminase (TG)

Zein-alginate-TG: Zein-alginate membranes prepared as described above were incubated at 4° C. in for 24 h in a MooGloo™ solution (TG), purchased from Modernist Pantry (Eliot, ME), (25% w/v) containing HEPES (0.1 M, pH 7.4) and CaCl₂) (15 g/L). 125 mL of MooGloo™ solution was used for each membrane. Afterwards each membrane was washed 2 times with 250 ml of HEPES (0.1 M, pH 7.4) containing CaCl₂) (15 g/L). Finally, the membranes were stored in a HEPES (0.1 M, pH 7.4) containing CaCl₂) (15 g/L) and 2× concentrated penicillin-streptavidin and antimycotic.

PPI-Alginate-TG: PPI-alginate membranes prepared as described above were incubated at 4° C. in for 24 h in a MooGloo™ (TG) solution (25% w/v) containing HEPES (0.1 M, pH 7.4) and CaCl₂) (15 g/L). 125 mL of MooGloo™ solution was used for each membrane. Afterwards, each membrane was washed 2 times with 250 ml of HEPES (0.1 M, PH 7.4) containing CaCl₂) (15 g/L). Finally, the membranes were stored in an ethanol/water solution 70/30% w/v.

Brown Rice-Alginate-TG: Brown rice-alginate membranes prepared as described above were incubated at 4° C. in for 24 h in a MooGloo™ (TG) solution (25% w/v) containing HEPES (0.1 M, pH 7.4) and CaCl₂) (15 g/L). 125 mL of MooGloo™ solution was used for each membrane. Afterwards, each membrane was washed 2 times with 250 ml of HEPES (0.1 M, pH 7.4) containing CaCl₂) (15 g/L). Finally, the membranes were stored in an ethanol/water solution 70/30% w/v.

Mung-Alginate-TG: Mung-alginate membranes prepared as described above were incubated at 4° C. in for 24 h in a MooGloo™ (TG) solution (25% w/v) containing HEPES (0.1 M, pH 7.4) and CaCl₂) (15 g/L). 125 mL of MooGloo™ solution was used for each membrane. Afterwards, each membrane was washed 2 times with 250 ml of HEPES (0.1 M, pH 7.4) containing CaCl₂) (15 g/L). Finally, the membranes were stored in an ethanol/water solution 70/30% w/v.

3.2 Thermal Crosslinking with Glycerol

### 3.2.1 Protein Membranes

The membrane changes from translucent to transparent as the water is exchanged throughout the porous structure. From there, the membrane is removed and placed into a third bath that is set to 130° C. for 10 minutes. Once the protein is crosslinked, the membrane is placed in the final bath that contains HEPES buffer at 7.4 pH to ensure the scaffold is at physiological pH for biological performance.

SPI: SPI flat sheet membranes were casted using a PTFE support sheet in a sodium acetate buffer (0.2 M, pH 4.5) and allowed to equilibrate in the same buffer for 10 minutes up to 3 hours. Afterwards, the PTFE supported membranes were transferred into a glycerol bath and allowed to exchange the water solution against glycerol over 10 min to 3 hours. Afterwards, membranes were thermally crosslinked either through a hot glycerol bath or by using an oven. In the first case, the membranes were transferred into a stirred glycerol bath at 100° C. and incubated for 10 minutes. Afterwards, different temperature ramps were investigated by varying the final temperature of the glycerin bath (between 110° C. and 140° C.) and temperature increments. In case of the oven treatment, membranes were incubated at different temperatures, ranging from 100° C. to 140° C., and for different time durations, from 10 to 24 hours.

Mung bean: Mung bean flat sheet membranes were casted using a PTFE support sheet into a sodium acetate buffer (0.2 M, pH 4.5) and allowed to equilibrate in the same buffer for 10 minutes up to 3 hours. Afterwards, the PTFE supported membranes were transferred into a glycerol bath and allowed to exchange the water solution against glycerol over 10 min to 3 hours. Afterwards, membranes were thermally crosslinked either through a hot glycerol bath or by using an oven. In the first case, the membranes were transferred into a stirred glycerol bath at 100° C. and incubated for 10 minutes. Afterwards, different temperature ramps were investigated by varying the final temperature of the glycerin bath (between 110° C. and 140° C.) and temperature increments. In case of the oven treatment, membranes were incubated at different temperatures, ranging from 100° C. to 140° C., and for different time durations, from 10 to 24 hours.

Wheat gluten: Wheat gluten flat sheet membranes were casted using a PTFE support sheet into a sodium acetate buffer (0.2 M, pH 4.5) and allowed to equilibrate in the same buffer for 10 minutes up to 3 hours. Afterwards, the PTFE supported membranes were transferred into a glycerol bath and allowed to exchange the water solution against glycerol over 10 min to 3 hours. Afterwards, membranes were thermally crosslinked either through a hot glycerol bath or by using an oven. In the first case, the membranes were transferred into a stirred glycerol bath at 100° C. and incubated for 10 minutes. Different temperature ramps were investigated by varying the final temperature of the glycerin bath (between 100° C. and 140° C.). In case of the oven treatment, membranes were incubated at different temperatures, ranging from 100° C. to 140° C., and for different time durations, from 10 hours to 24 hours.

### 3.2.2 Protein-Alginate Membranes

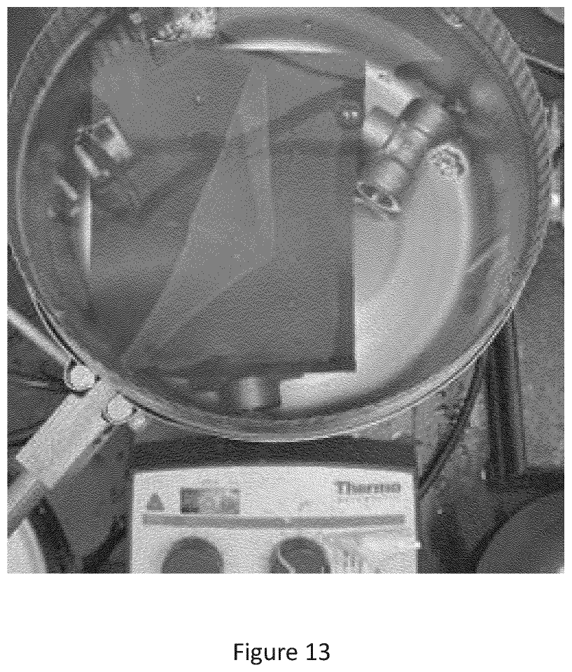

Mung bean-alginate: Mung bean-alginate flat sheet membranes were casted using a PTFE support sheet into a sodium acetate buffer (0.2 M, pH 4.5) containing CaCl₂) (15 g/L), and allowed to equilibrate in the same buffer for 10 minutes up to 3 hours. Afterwards, the PTFE supported membranes were transferred into a glycerol bath and allowed to exchange the water solution against glycerol over 10 min to 3 hours. Afterwards, membranes were thermally crosslinked either through a hot glycerol bath or by using an oven. In the first case, the membranes were transferred into a stirred glycerol bath at 100° C. and incubated for 10 minutes. Afterwards, different temperature ramps were investigated by varying the final temperature of the glycerin bath (between 110° C. and 140° C.) and temperature increments. In case of the oven treatment, membranes were incubated at different temperatures, ranging from 100° C. to 140° C., and for different time durations, from 10 to 24 hours. See, FIG. 13.

Wheat gluten-alginate: Wheat gluten-alginate flat sheet membranes were casted using a PTFE support sheet into a sodium acetate buffer (0.2 M, pH 4.5) containing CaCl₂) (15 g/L), and allowed to equilibrate in the same buffer for 10 minutes up to 3 hours. Afterwards, the PTFE supported membranes were transferred into a glycerol bath and allowed to exchange the water solution against glycerol over 10 min to 3 hours. Afterwards, membranes were thermally crosslinked either through a hot glycerol bath or by using an oven. In the first case, the membranes were transferred into a stirred glycerol bath at 100° C. and incubated for 10 minutes. Afterwards, different temperature ramps were investigated by varying the final temperature of the glycerin bath (between 110° C. and 140° C.) and temperature increments. In case of the oven treatment, membranes were incubated at different temperatures, ranging from 100° C. to 140° C., and for different time durations, from 10 to 24 hours.

Zein-alginate: Zein-alginate flat sheet membranes were casted using a PTFE support sheet into a sodium acetate buffer (0.2 M, pH 4.5) containing CaCl₂) (15 g/L), and allowed to equilibrate in the same buffer for 10 minutes up to 3 hours. Afterwards, the PTFE supported membranes were transferred into a glycerol bath and allowed to exchange the water solution against glycerol over 10 min to 3 hours. Afterwards, membranes were thermally crosslinked either through a hot glycerol bath or by using an oven. In the first case, the membranes were transferred into a stirred glycerol bath at 100° C. and incubated for 10 minutes. Afterwards, different temperature ramps were investigated by varying the final temperature of the glycerin bath (between 100° C. to 110° C. and 100° C. to 140° C.). In case of the oven treatment, membranes were incubated at different temperatures, ranging from 100° C. to 140° C., and for different time durations, from 10 to 24 hours.

### 4 Membrane Coatings

### 4.1 Bovine Collagen Coating (Method 1)

Mung bean membranes were coated with bovine collagen to increase their affinity to cell promoting cell adhesion and proliferation. Dry, 14 mm-diameter mung bean membrane discs were soaked in a 3 mg/mL collagen solution (20 discs per 20 mL collagen solution) for two hours at room temperature. Afterwards, the collagen solution was removed, and the discs were put in a 100% ethanol solution and stored at 4° C. prior to use.

### 4.2 Bovine Collagen Coating (Method 2)

Mung bean membranes were coated with bovine collagen to increase their affinity to cell promoting cell adhesion and proliferation. Dry 14 mm-diameter mung bean membrane discs were soaked in a 3 mg/ml collagen solution (20 discs per 20 mL collagen solution) for two hours at room temperature. Afterwards, the collagen solution was removed, and the discs were incubated in a HEPES solution (0.1 M, pH 7.4) for 1 hour at 37° C. Afterwards, the HEPES solution was removed, and the discs were stored in a 70/30 w/v ethanol-water solution at 4° C. prior to use.

### 4.3 Bovine Fibronectin Coating (Method 1)

Mung bean membranes were coated with bovine fibronectin to increase their affinity to cell promoting cell adhesion and proliferation. Dry 14 mm-diameter mung bean membrane discs were soaked in a 2.5 mg/ml fibronectin solution (20 discs per 20 ml fibronectin solution) for two hours at room temperature. Afterwards, the fibronectin solution was removed, and the discs were put in a 100% ethanol solution and stored at 4° C. prior to use.

### 4.4 Chitosan Coating

Mung bean membranes were coated with chitosan to increase their affinity to cell promoting cell adhesion and proliferation. Dry 14 mm-diameter mung bean membrane discs were soaked in a 1% w/v chitosan acetic acid solution (0.2 M, pH 4.5, 20 discs per 20 mL chitosan solution) for one hour at room temperature. Afterwards, the chitosan solution was removed, and the discs were put in a 10% TPP solution and agitated for 3 hours. Afterwards, the discs were washed 2× with MilliQ™ water and stored in 70/30 w/v at 4° C.

### Storage:

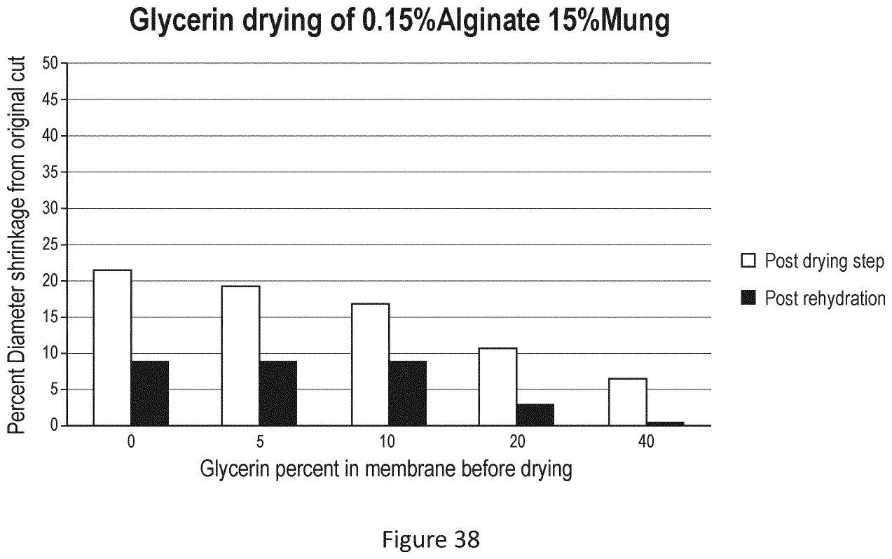

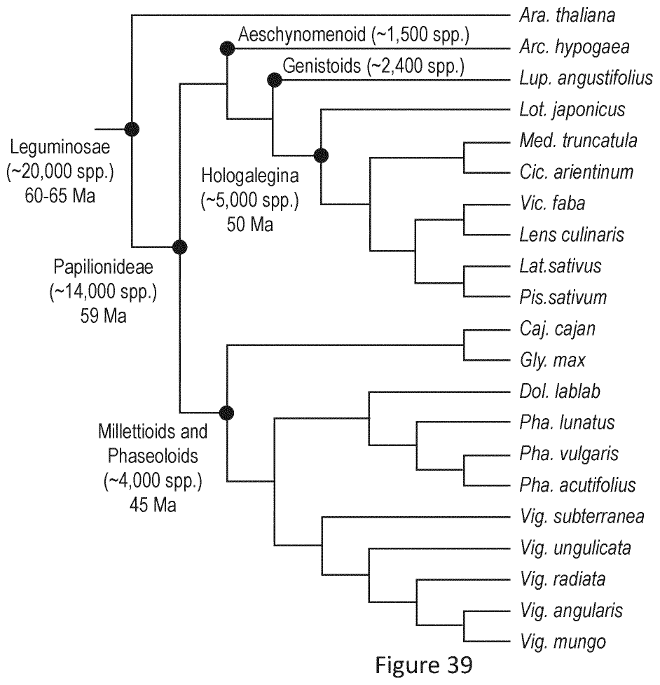

The membranes can be stored in 70/30 ethanol/MilliQ™ w/v OR HEPES with antibiotic/antimycotic. Or if can be dried, but attention to pore collapse must be considered. Drying can be accomplished with freeze drying equipment. More scale-able and flexible membrane can be dried if another exchange bath consisting of water and 20-40% glycerin is used to exchange out the HEPES. If the pores of the membrane are filled with the 20-40% glycerin, then the porous structure can be dried. See, FIG. 38.

### 5. Mechanical Studies on Membranes

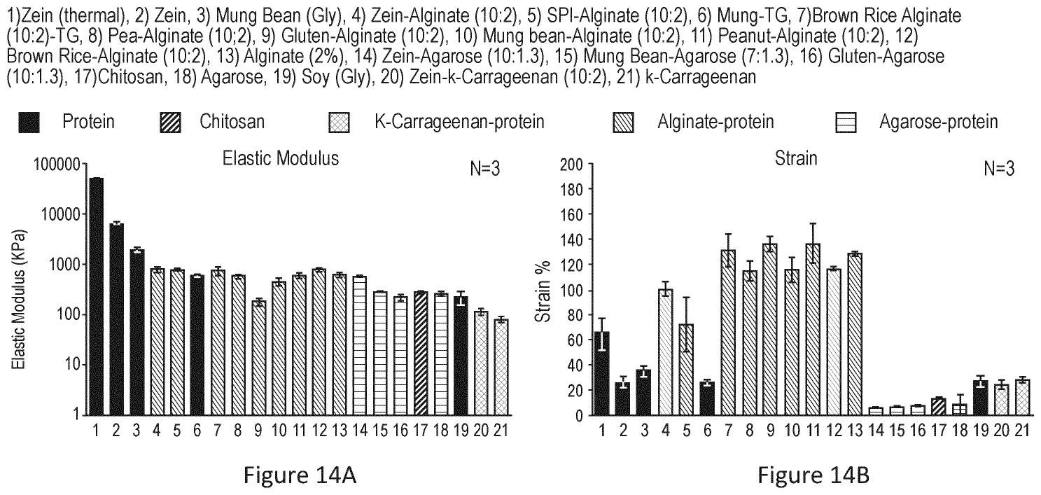

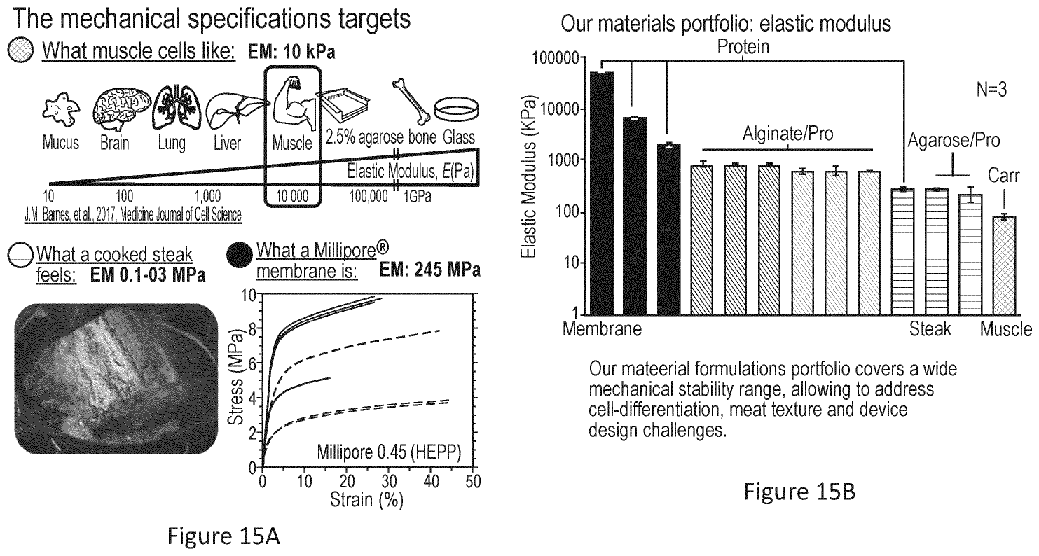

The membrane mechanical properties were characterized in tensile mode using a ZwickRoel tester. As shown in FIG. 14, the elastic moduli of the membranes cover a wide range of values, enabling our material portfolio to comprehensively address the diverse design specifications for the hollow fibers. For instance, k-carrageenan-based membranes have elastic moduli below the 100 kPa, and therefore suitable as substrates for muscle cell growth and differentiation (See, FIG. 15). As the hollow fibers becomes part of the final cultured meat product, the textural profile of real meat also needs to be considered as design specification for our materials. To this regard, we designed thermally threated soy, agarose-blends and some alginate blends falling in the 100-300 kPa elastic modulus range, which is known to be characteristic for meat, in particular a whole cut steak. The highest mechanical performances in terms of elastic modulus and strain to break are achieved with pure proteins, like mung mean and zein, or alginate-protein blends. These last materials can be used as structural components allowing the hollow fibers to undergo a wide range of fabrication processes and finally sustain the working conditions when installed in the bioreactor.

### Results

### 5.1 Optimization of the Glycerol Method

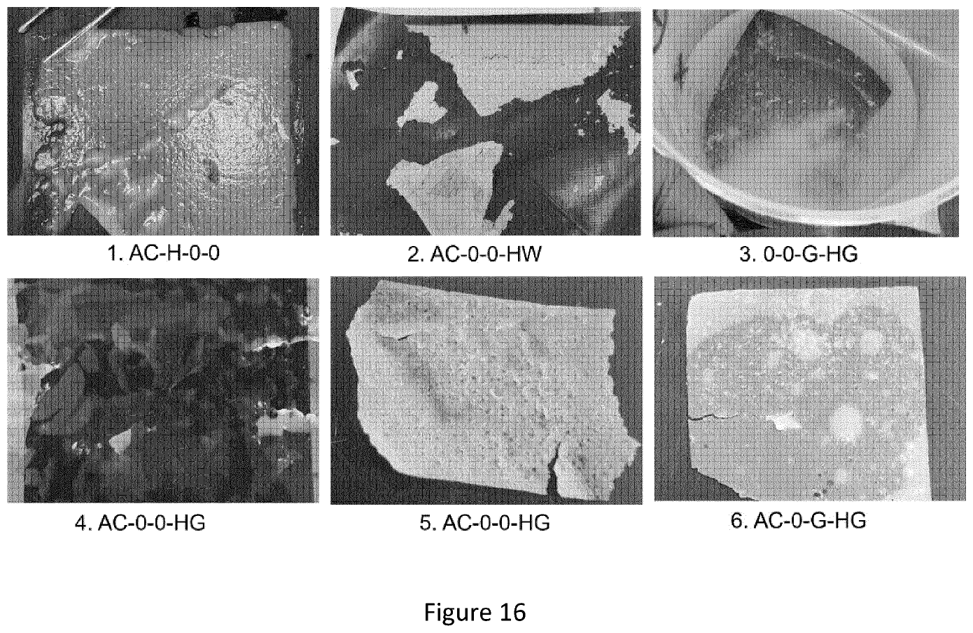

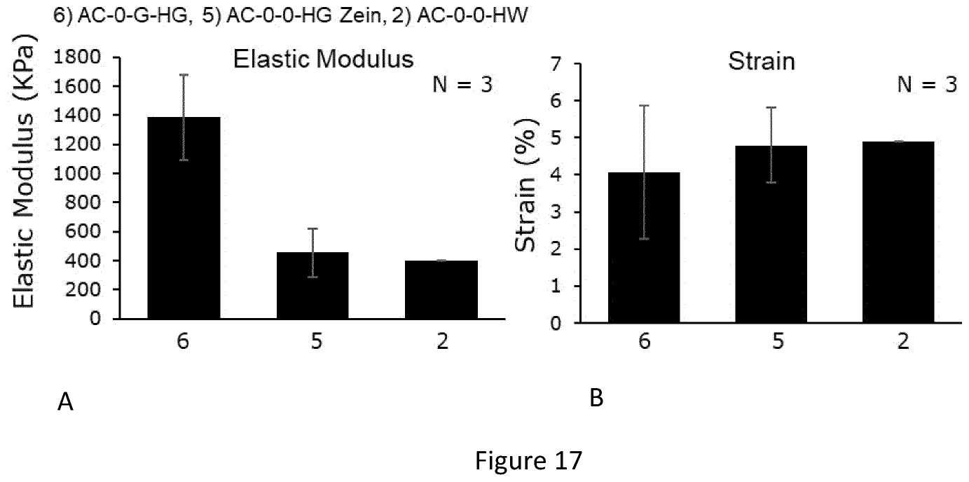

The final process for the glycerol crosslinking, comprising of the sequential coagulation (1), neutralization (2), glycerin-water exchange (3) and glycerin thermal treatment (4) steps was validated by testing the effect of each step as shown in table 1. After coagulation in acetate and neutralization in HEPES, the absence of the glycerol heat treatment (sample 1, AC-H-O-O) leads to membranes which are mechanically instable and have a paste consistency (See, FIG. 16). Similarly, replacing the glycerol treatment with autoclaving (121° C.) leads to unstable and brittle membranes (sample 4 AC-O-O-HW). Powder-like and mechanical unstable membrane were also obtained when the initial acetate coagulation and neutralization steps were removed and only the glycerol heat treatment was applied (Sample 3 O-O-G-HG) (See, FIG. 16). This emphasizes the importance of having a coagulated protein network essential for the stability of the membrane. Also, if the coagulation does not happen in acidic condition but rather neutral condition (HEPES), a very brittle membrane is obtained (Sample 4 O-H-G-HG). Finally, exchanging the water with glycerol at room temperature before the heat treatment helps avoid the formation of large bubbles resulting from the sudden expansion of water when in contact with the heated glycerol bath (sample 5 AC-O-O-HG). As a result, the best membranes were obtained by coagulating the dope solution using an acetate bath, exchanging the water with glycerol at room temperature and finally thermally cross-linking the protein network making use of a heated glycerol bath (sample 6 AC-O-G-HG). Compared to the other membrane samples, the one obtained according to the AC-O-G-HG lead to the most stable membranes having the highest young modulus and lowest strain, indicating a higher degree of protein cross-linking (See, FIG. 17).

| | 1^{st} formation | Neutralization | Glycerin Bath | Glycerin Heating | |
|---|---|---|---|---|---|
| | (Acetate - | (HEPES - | (room temp for | (120° for | Working |
| Sample | 10 mins) | 2 hours) | 10 mins) | 10 mins) | Label¹ |
| 1 | Y | N | Y | Y | AC-0-G-H [symbol] |
| 2 | Y | N | N | Y | AC-0-0-H [symbol] |
| 3 | Y | Y | N | N | AC-H-0- [symbol] |
| 4 | Y | N | N | water² | AC-0-0-H [symbol] |
| 5 | N | N | Y | Y | 0-0-G-H [symbol] |
| 6 | N | Y | Y | Y | 0-H-G-H [symbol] |

¹AC = acetate, H = Hepes, G = glycerin, HG = hot glycerin, 0 = step not performed.
²hot water treatment: autoclave 121° C. for 60 mins [symbol]
indicates data missing or illegible when filed

Table 1: Experimental conditions for the optimization of the glycerol crosslinking method. AC stands for “Acetate bath 0.2 M at pH 4.5”, H stands for “HEPES bath 0.1 M at pH 7.4”, G stands for “glycerol bath”, HG stands for “hot glycerol bath”, HW stands for “hot water treatment” (autoclave 121° C.); 0 stands for “step not performed”; Y stands for “yes”, and; N stands for “no”.

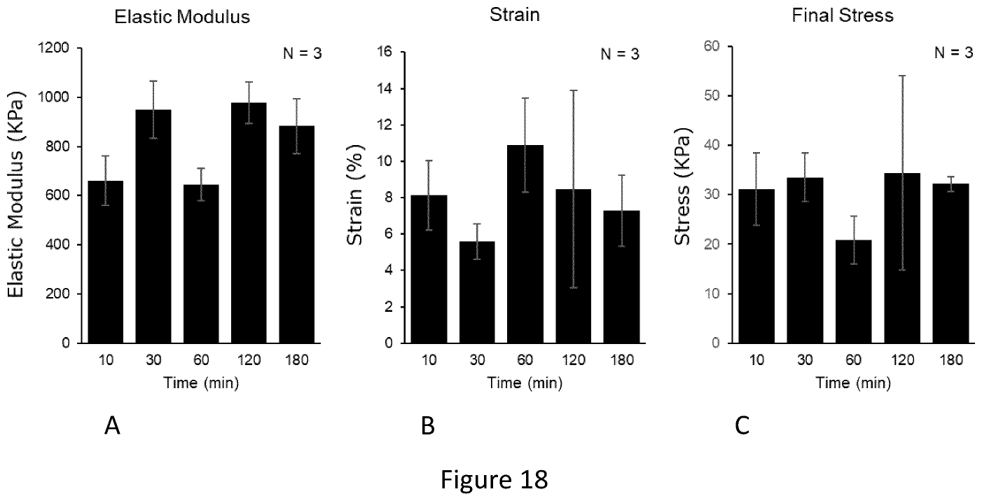

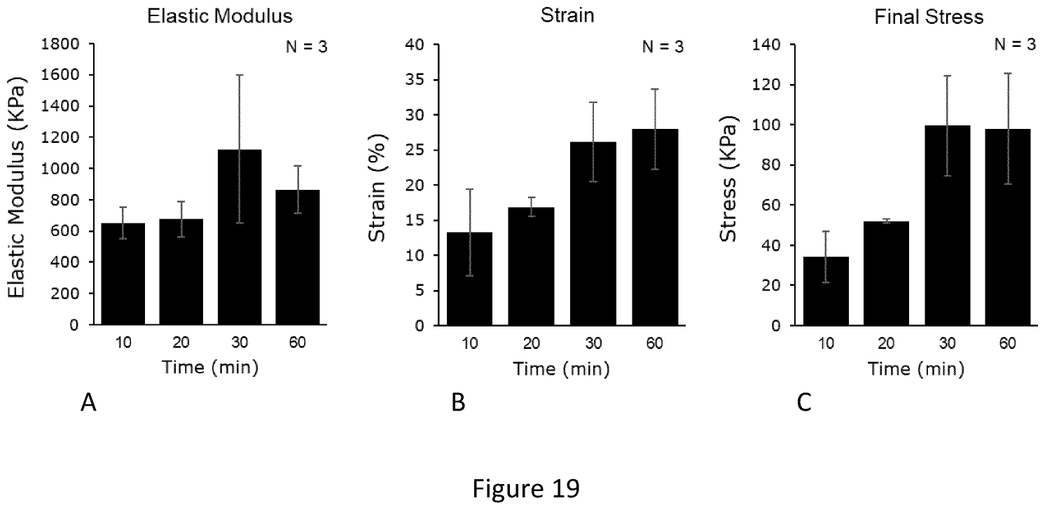

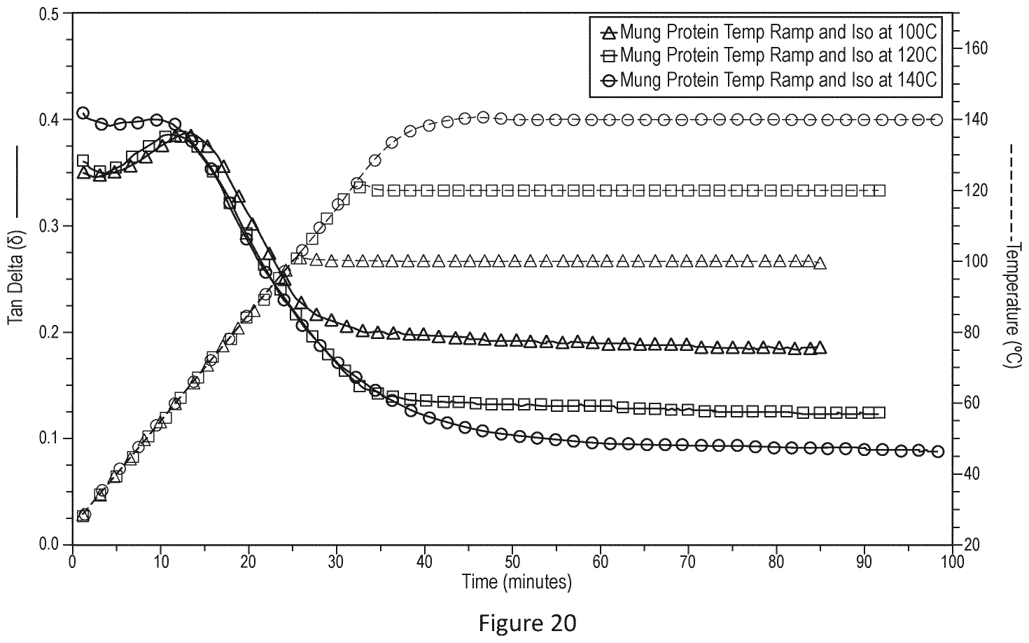

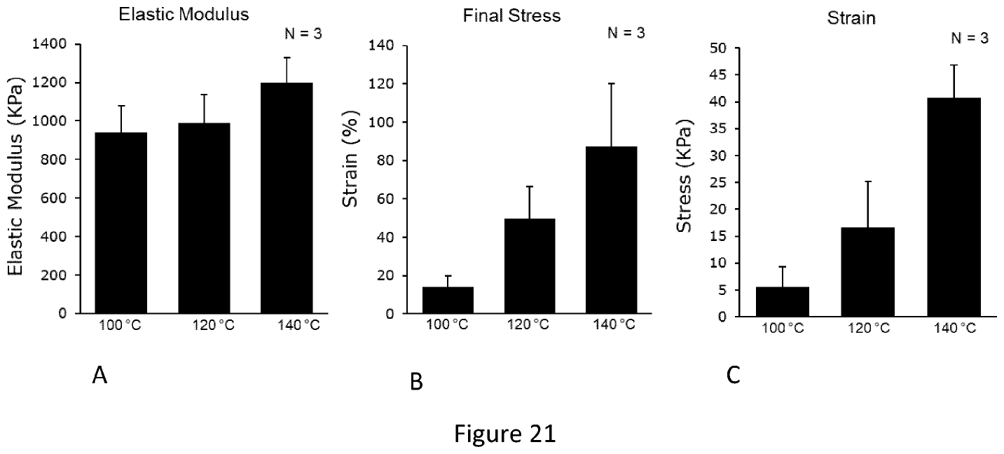

Each step of the glycerol-based thermal treatment was further optimized to improve membrane morphology and mechanical properties. The effect of the acetate coagulation step was investigated by varying the acetate bath duration and keeping constant both the water-glycerol exchange (10 minutes) and glycerol-based heat treatment (temperature ramp: 10 minutes at 100° C., ramp to 120° C. and 30 minutes at 120° C.) conditions. FIG. 18 shows the mechanical properties of the membranes coagulated for 10 minutes up to 3 hours. No statistical differences for the elastic modulus, final strain and final stress are observed upon increase of coagulation time, indicating that the coagulation is complete within the 10 minute window explored. These results suggest that 10 minutes are sufficient to allow for the neutralization of the membrane pH, thus a successful coagulation process. Next the glycerol-based heat treatment was investigated by keeping constant the duration of both coagulation bath (10 mins) and water-glycerol exchange (10 mins) and varying the heat treatment duration after reaching the final temperature of 120° C. As shown in FIG. 19, stronger and tougher membranes are obtained upon increase of heat treatment time, with final strain and stress doubling and triplicating in value, respectively. It is also noticed that, after 30 minutes the membranes mechanical properties start plateauing, with almost no difference in final stress between the 30 mins and the 60 mins samples. As the heat treatment seemed to have the larger effect on the mechanical properties of the membranes, further investigation was carried to evaluate the effect of the final temperature of the ramp. This time, the change in physical properties of the membrane was monitored using rheological analysis. The heat treatment was directly performed in the rheometer chamber on membranes which were first coagulated (10 mins) and had undergone water-glycerol exchange (10 mins). As shown in FIGS. 20 & 21, membranes were subjected to a heat ramp of 4 degree per minute starting from 20° C. and let equilibrate at three different final temperatures of 100° C., 120° C. and 140° C. At 50-60° C., tan (8) starts decreasing, thus suggesting the initiation of the protein annealing process leading to the membrane solidification. Heat-driven protein unfolding and formation of interchain physical crosslinks is believed to be the mechanism for the solidification process. Interestingly, a trend in tan (8) is observed upon change of final temperature of the isothermal ramp. Lower values of tan (8) are obtained over increase of the ramp final temperature, thus suggesting the membrane to undergo a strengthening process upon increase of the annealing temperatures. This trend was confirmed by tensile tests carried out on the samples obtained from the rheology experiments. As shown in FIG. 20, an increase of elastic moduli, final stress and strain is observed upon increase of final isothermal temperature.

The membrane structure formation starts around 50-60° C. and continues to form at the same rate irrespective their final isothermal temperature. However, the final, resulting membrane structure strength appears to be affected by the final isothermal condition. The higher isothermal temperature results membrane with more elasticity (lower tan (8))

### 6. Stability Tests in Cell Media

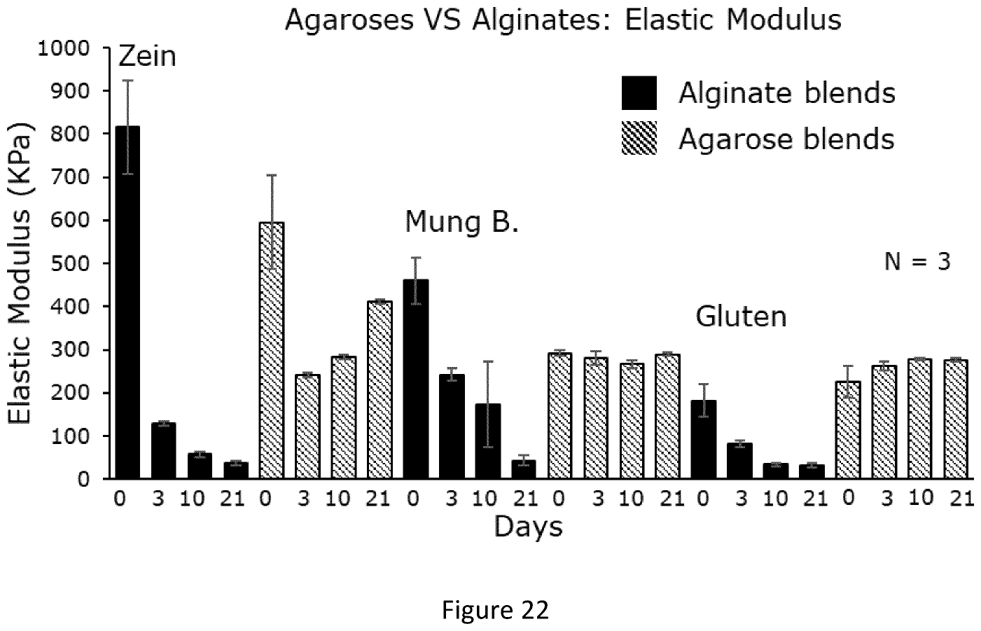

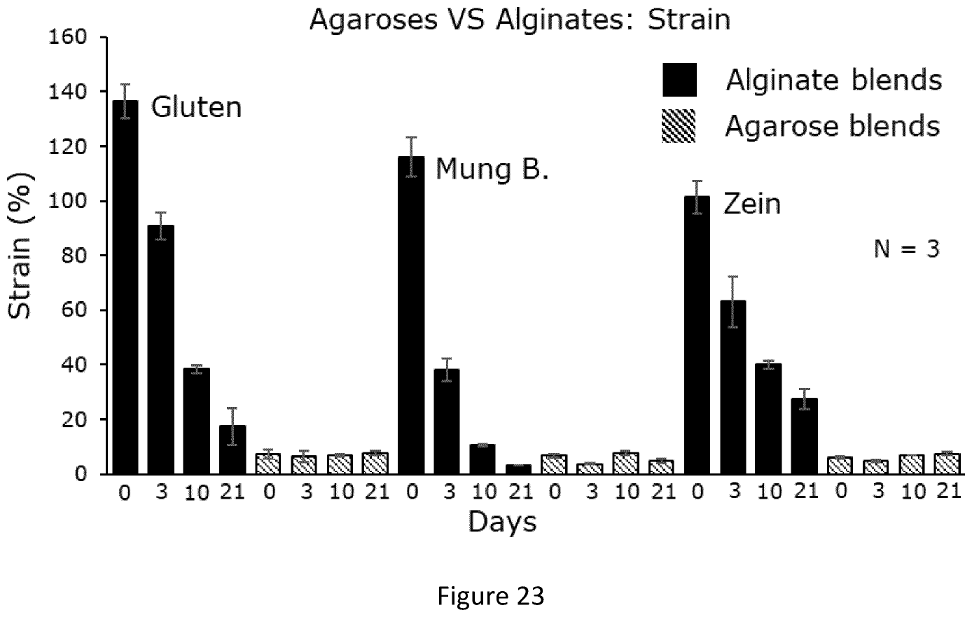

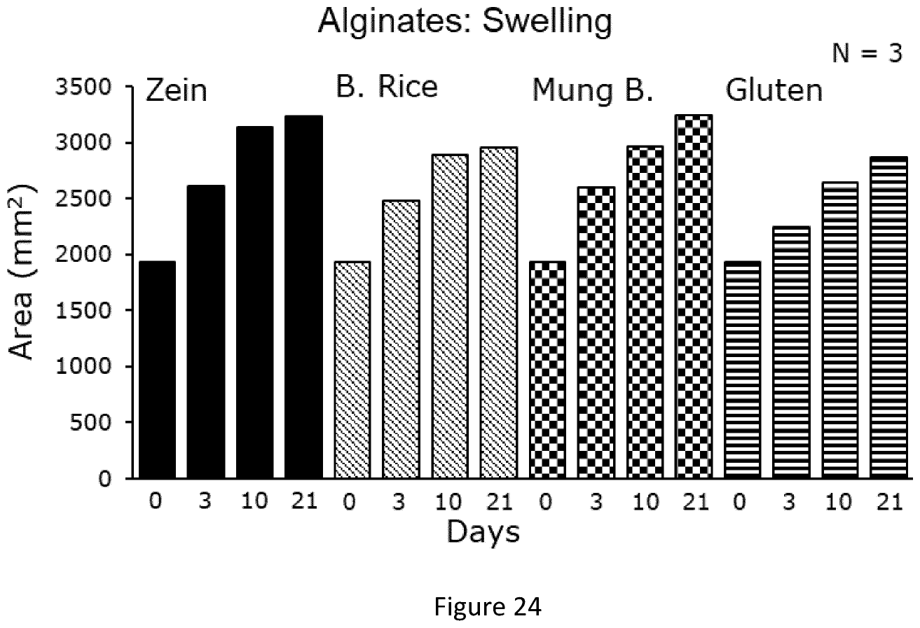

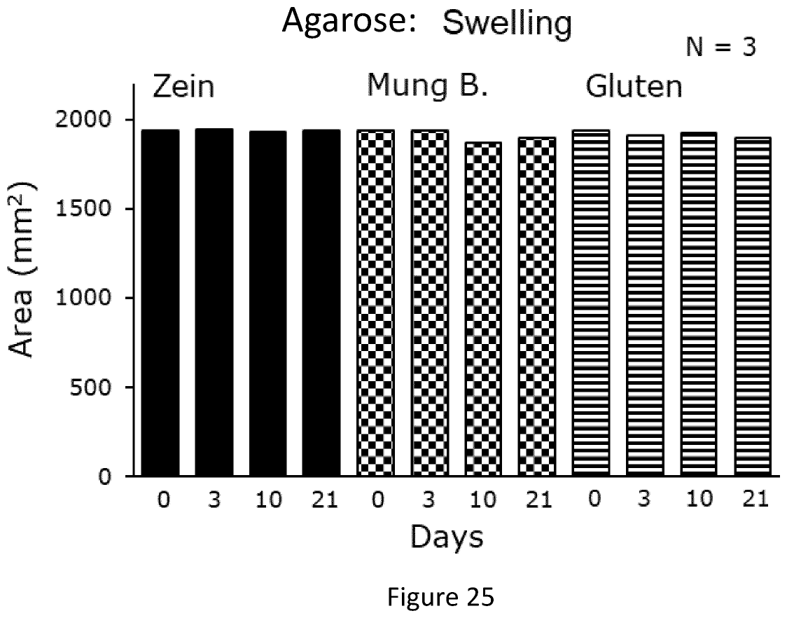

To test the stability of the materials under cell-culture conditions, membranes were incubated in cell media at 37° C. up to 30 days and mechanical tests were performed at different time points to investigate their integrity. K-carrageenan and its pea protein isolate blend turned out to be highly unstable in cell culture media, undergoing full dissolution already after 1 day of incubation. Differently, alginates and agarose blends were found to be more stable over a longer incubation time. In the latter case, the performances of the membranes are believed to be mainly affected by the stability of the alginate and agarose polysaccharide components. This observation is supported by the presence of two distinct stability trends depending on the nature of the polysaccharide. Alginate blends undergo a dramatic decrease in both elastic modulus and strain, with the zein blend undergoing a decrease in elastic modulus of more than 10 fold. In contrast, agarose blends preserve their mechanical properties almost entirely throughout the whole incubation period of 21 days. See, FIGS. 22, 23, 24 and 25.

In case of alginate blends, the gradual decease in mechanical stability was believed to be caused by the decomplexation of the calcium-glutaric acid crosslinking polymer network. This hypothesis is supported by the noticeable swelling behavior of the membranes upon incubation time, which was quantified as increase in the membrane surface area (FIG. 22). In contrast, no swelling was observed for the agarose blend-based membranes. The correlation between the swelling and mechanical stability trends indicates the polysaccharides network to be the main structural component of the membrane, which, in case of alginate, was subject to failure under culturing condition.

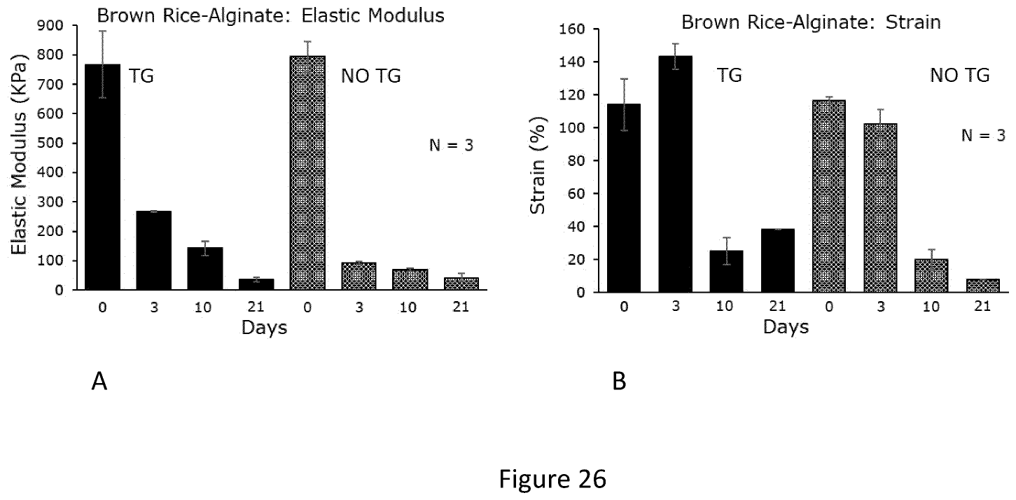

To increase the stability of the alginate-protein blends in cell-culture conditions, crosslinking of the protein component was investigated. Transglutaminase was chosen as first crosslinking candidate to test, as it is commonly used in the food industry for the preparation of processed meat. As shown for the brown rice-alginate blend, also in this case, decrease in both elastic modulus and strain were observed upon increase of incubation time. See, FIG. 26.

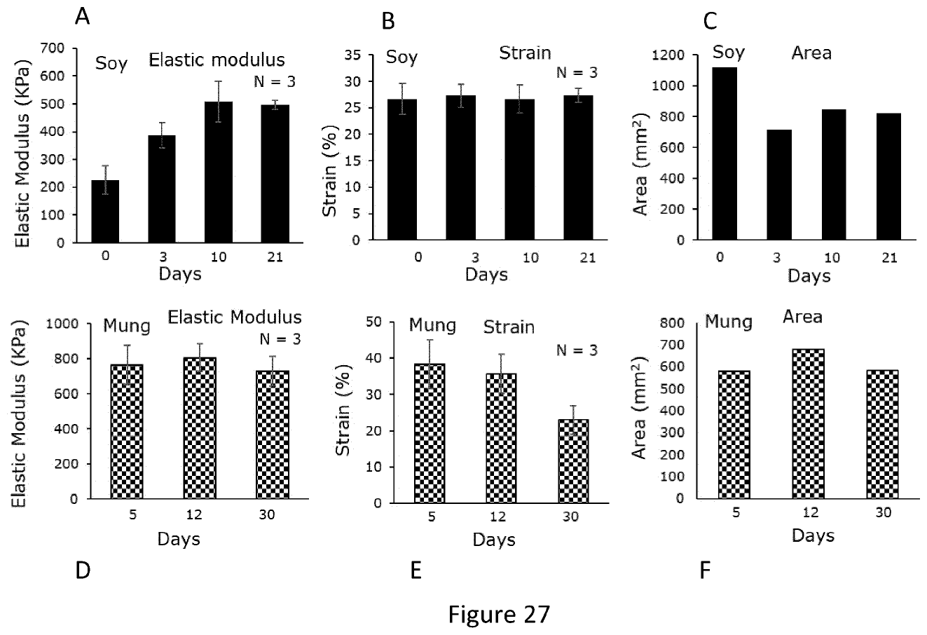

Thermal annealing was chosen as alternative approach to induce physical crosslinking of the protein polymer network and ultimately stabilize the membrane during cell culture. To avoid the collapsing of the membrane porous structure formed via phase reverse transition, glycerol was used both as water exchange medium and also heat transfer vector for the annealing process. Compared to the alginate blends, both thermally annealed soy and mung bean do not show a decrease in elastic modulus when incubated in cell media at 37° C. After 21 days, soy membranes underwent an increase in elastic modulus almost doubling its value. The strain to break (elongation at break) was unaffected, while, in case of soy, a slight decrease in surface area suggested a possible further crosslinking process occurring over time. A slight decrease into the force needed to cause a break was observed for mung bean after 30 days of incubation. The higher mechanical stability in cell culture condition compared to alginate-protein blends and the higher strain to break compared to agarose-protein blends, make these heat-treated pure protein materials the preferred candidates for the development of membranes for bioreactor applications. See, FIG. 27.

### 7. Imaging Porosity

### 7.1 Flat Sheet Membranes

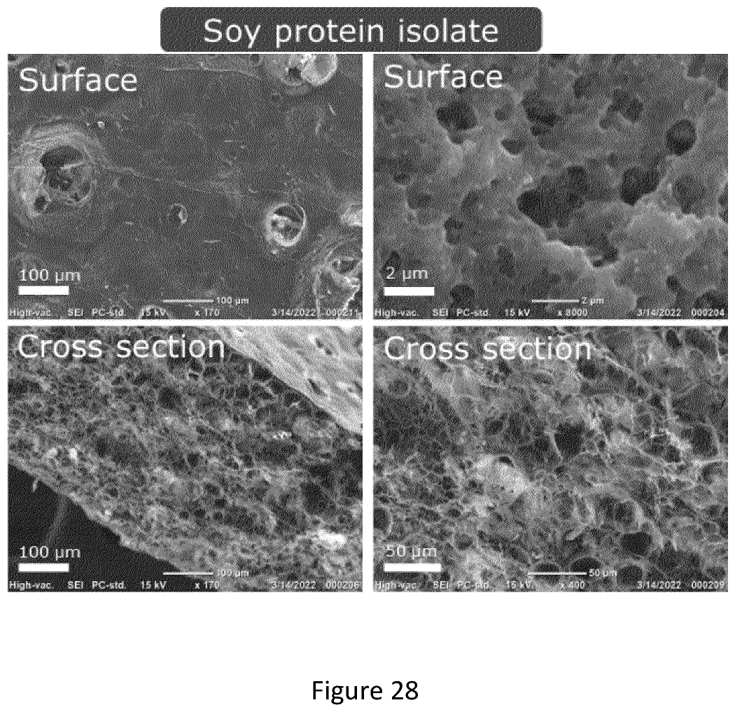

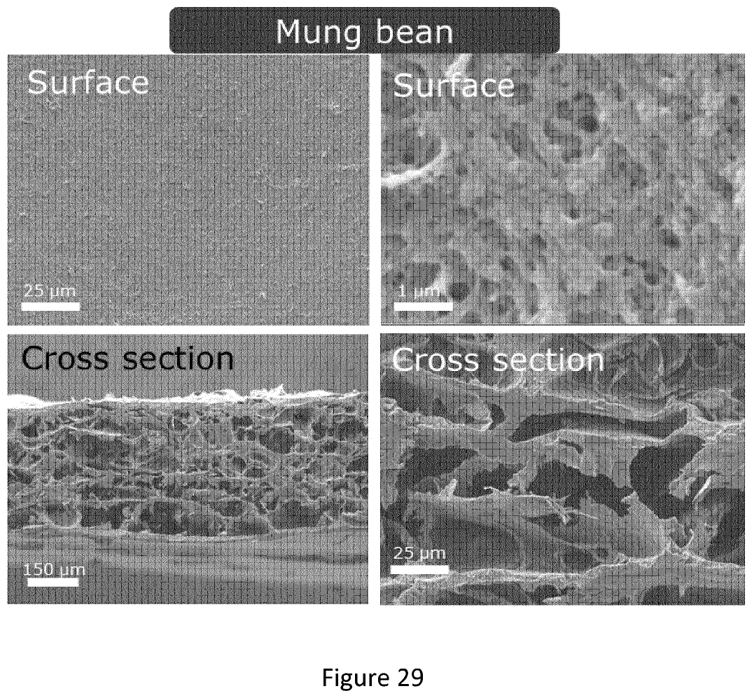

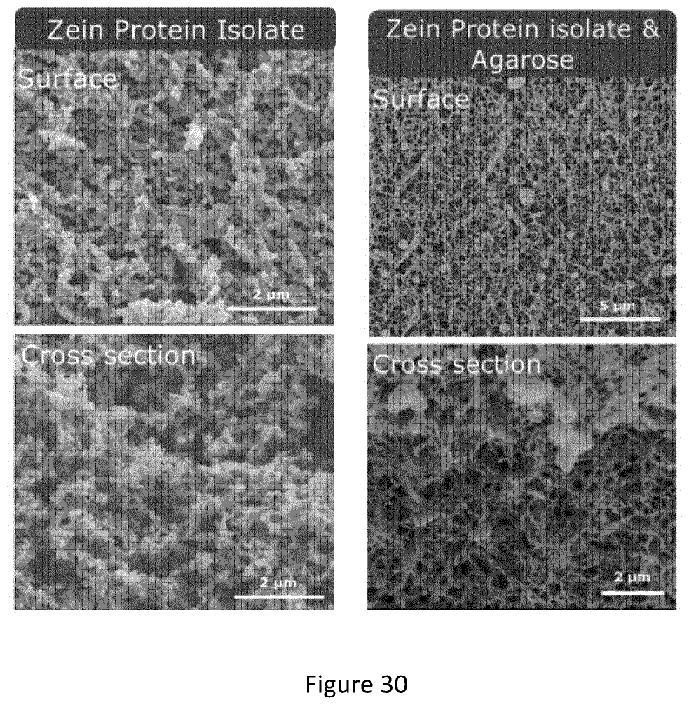

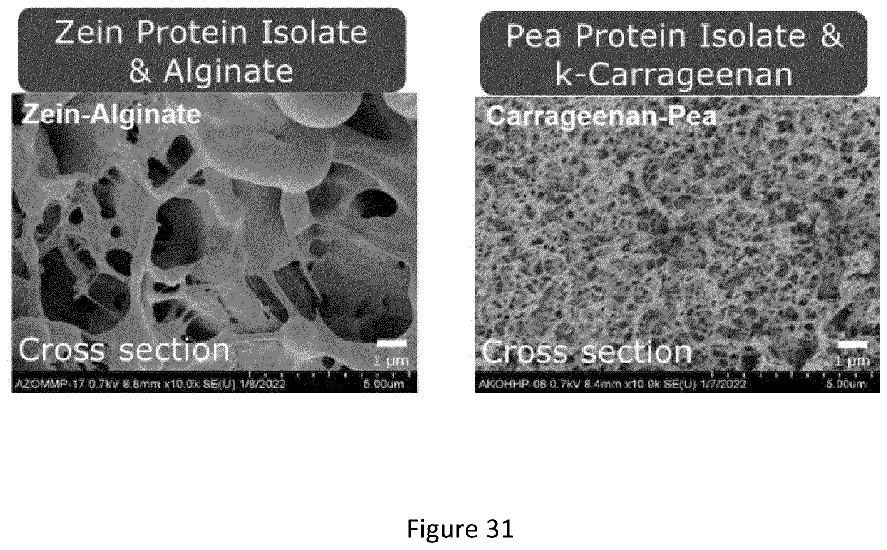

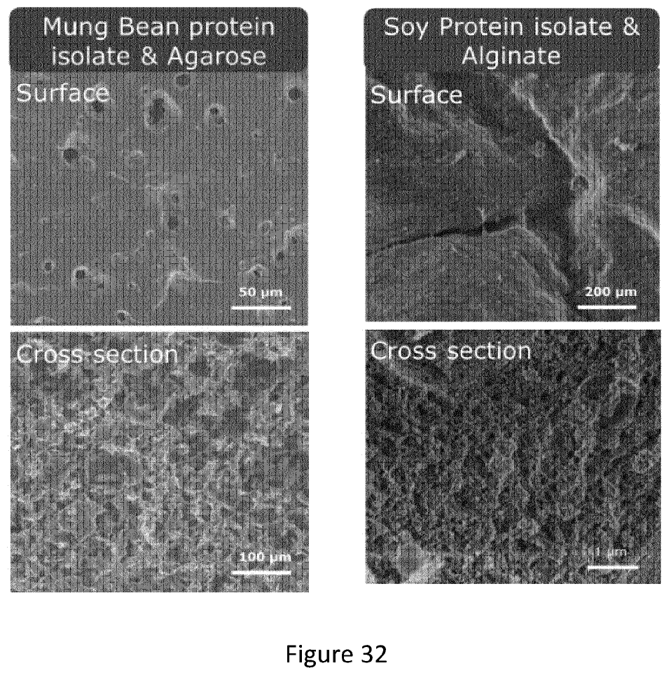

The porosity of the membranes produced was investigated via scanning electron microscopy. As shown in FIGS. 28 and 29, respectively, heat-treated soy and mung bean protein membranes present an heterogenous porosity, which is characterized by smaller pores in the submicron range on the surface and larger pores in the 20-50-micron range located in the cross section. A fast coagulation process occurring at the membrane-bath solution interface during the coagulation process is believed to give origin to the thinner porosity located on the surface. In contrast, a slower coagulation process occurring in the core of the membrane allows for a greater phase separation leading to larger pores. A different scenario is observed in case of zein and agarose-zein, where a homogeneous porosity is observed throughout the whole membrane. FIG. 30 shows, in this latter case, the phase separation process was the result of a fibrillation process leading to a very homogeneous pore size distribution. While the present invention is not limited by theory, it is hypothesized that both agarose and zein are known to undergo fibrillation via protein self-assembly. A similar result was observed in case of alginate-zein and pea-k-carrageenan membranes (see, FIG. 31), where the biopolymer fibrillation was also the leading process for membrane formation. In contrast, a skinning effect was observed for mung bean-agarose and soy-alginate membranes. See, FIG. 32.

### 7.2 Hollow Fiber Membranes

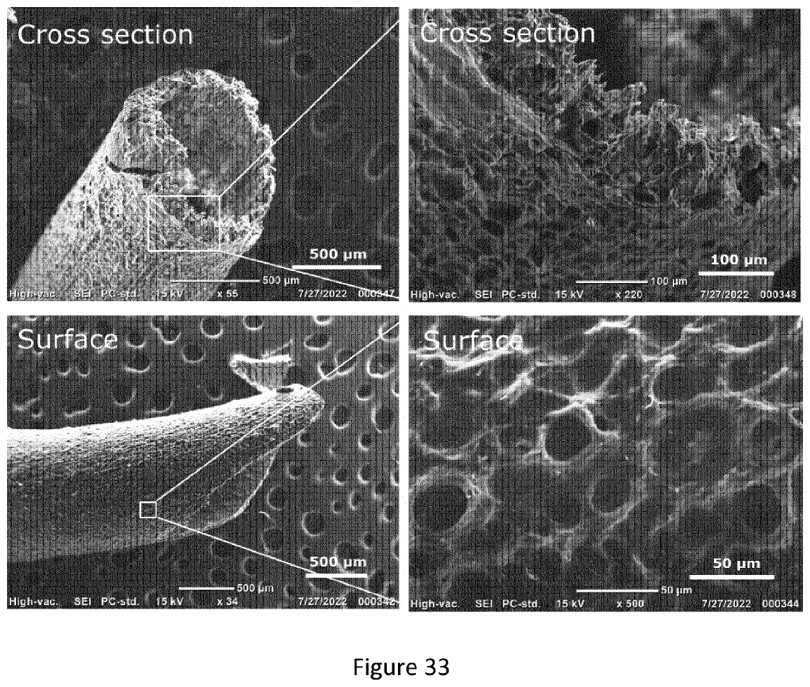

The porosity of the hollow fibers was investigated using a scanning electron microscope. FIG. 33 shows the cross section (top) and surface (bottom) of a mung bean-alginate (15%-0.2%) hollow fiber. The fiber presents pores in the 50-micron range and below throughout the whole cross section, while no skinning effect was observed. The fiber wall thickness was in the 100-micron range, value which has been targeted to optimize the outer nutrient diffusion considering the theoretical diffusion typically observed in tissue with thicknesses greater than 200 microns.

### 8. Cell Adhesion and Proliferation Studies

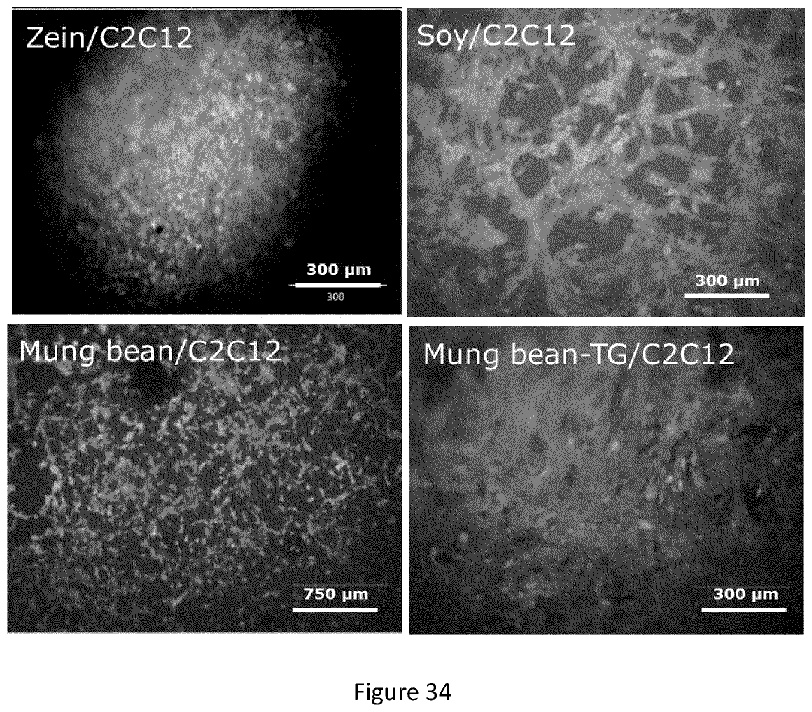

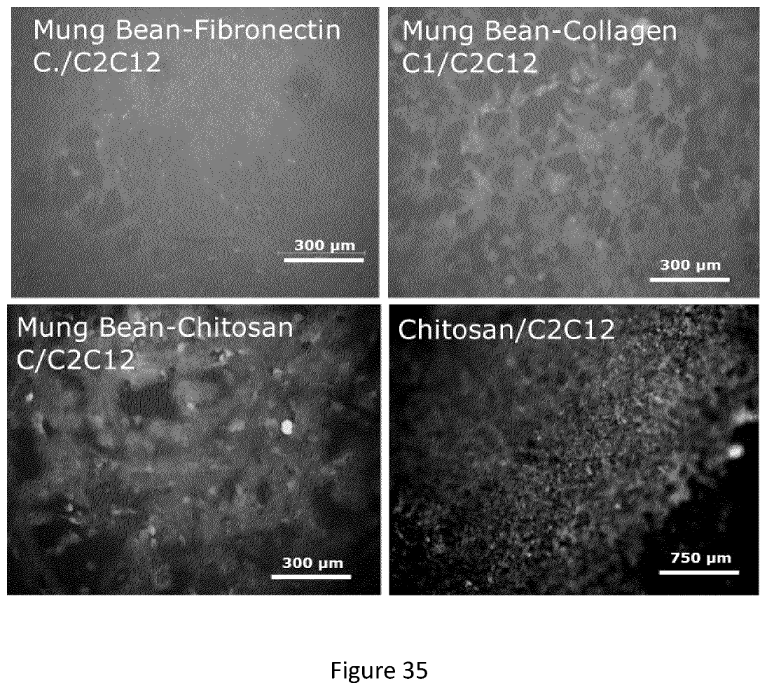

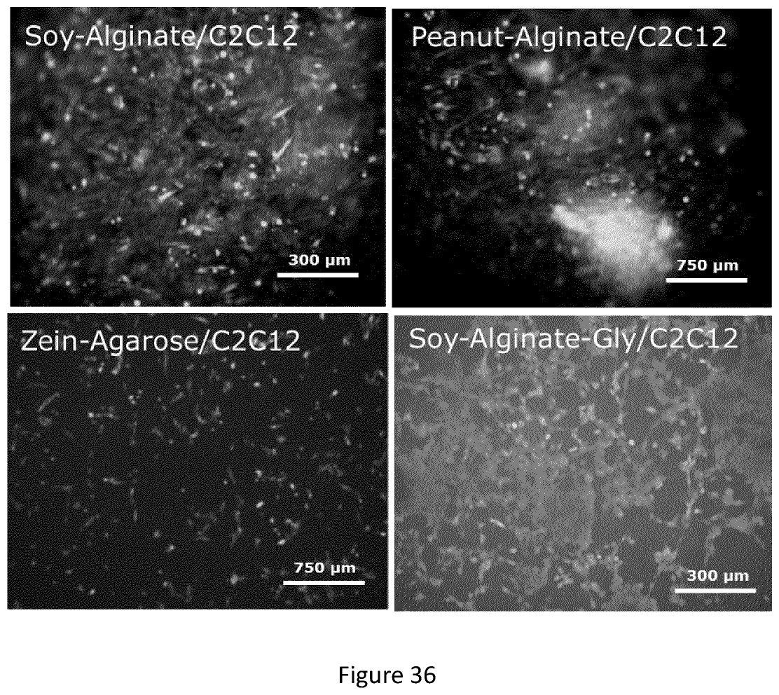

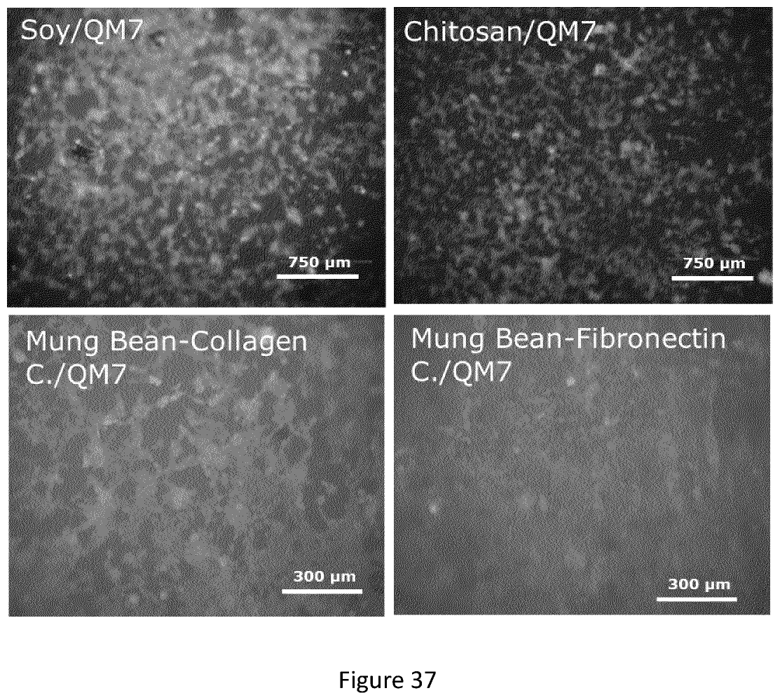

The produced membranes were tested for cell adhesion and proliferation using C2C12 (see, FIGS. 34, 35 and 36) and QM7 (see, FIG. 37) cell lines. Generally, higher degrees of adhesion and proliferation were obtained in case of pure protein membranes, an observation that was supported by the presence of cells with a more elongated morphology both in case of C2C12 and QM7. The best results were achieved when the protein membranes were coated with cell-adhesion proteins such as collagen and fibronectin. Differently, a more spherical and cluster-like assembled cells were found in case of protein-polysaccharides blends, indicating a lower affinity of the material for both C2C12 and QM7 cell lines.

1. A method for manufacturing cross-linked, edible, porous hollow fibers or sheet membranes, comprising:
a) providing: i) one or more edible proteins, ii) one or more solvents iii) a formation bath; wherein the one or more solvents or the formation bath also comprise one or more multivalent cations or anions or a buffer solution;
b) co-mixing the one or more edible proteins in the one or more solvents to form a mixture;
c) extruding the mixture into the formation bath to form an extruded hollow fiber or casting the mixture into the formation bath to form a sheet membrane; and
d) exposing the extruded hollow fiber or sheet membrane to an energy source selected from one or more of heat and irradiation sufficient to at least partially crosslink the one or more proteins to form cross-linked, edible, porous hollow fibers or sheet membrane.

2. The method of claim 1, further providing one or more edible polysaccharides and, in step b), co-mixing the one or more polysaccharides with the one or more edible proteins in the one or more solvents.

3. The method of claim 1, further providing a plasticizer and, in step b) co-mixing the plasticizer with the one or more edible proteins in the one or more solvents.

4. The method of claim 1, wherein the one or more proteins are selected from a group consisting of pea, soy, wheat, pumpkin, rice, brown rice, sunflower, canola, chickpea, lentil, mung bean, navy bean, corn, oat, potato, quinoa, sorghum and peanut.

5. The method of claim 2, wherein said one or more polysaccharides are selected from a group consisting of agar, chitosan, chitin, alginate, sodium alginate, cellulose, hydroxypropyl cellulose, Methyl cellulose, hydroxypropyl methylcellulose, gellan gum, xanthan gum, pectin, tapioca, guar gum and bean gum.

6. The method of claim 1, wherein said one or more solvents are selected from a group consisting of water, acetic acid, citric acid, lactic acid, phosphoric acid, malic acid, tartaric acid, sodium hydroxide, ethanol, glycerin and propylene glycol.

7. The method of claim 1, wherein said formation bath comprises one or more of calcium, zinc, magnesium, iron and potassium, in combination with one or more of 1) water, acetic acid, citric acid, lactic acid, phosphoric acid, malic acid, tartaric acid, or one or more of 2) sodium hydroxide and potassium hydroxide.

8. The method of claim 1, wherein said ion is selected from the group consisting of Ca2+, Mg2+, Fe3+, Zn2+, tripolyphosphate and trisodium citrate and wherein said selected ion is capable of at least enabling partial crosslinking of the one or more polysaccharides.

9. The method of claim 1, wherein said heat in step d) is from about 70° C. to about 140° C., applied under a pressure of from about 0 PSI to about 20 PSI gauge, at a relative humidity of from about 50% to about 100%, for about 2 to about 60 minutes or the hollow fiber or sheet membrane is dipped in a water bath that is from about 60° C. to about 100° C. at atmospheric conditions.

10. The method of claim 1, wherein the mixture of step b) is heated.

11. The method of claim 1, wherein the co-mixing of step b) is performed at about 0° C. to about 90° C.

12. The method of claim 1, wherein said mixture is at a pH of about 10 to about 13 and said formulation bath is at a pH of about 3 to about 5.

13. The method of claim 12, wherein after formation said membrane is neutralized to a pH of about 6.8 to about 7.8.

14. The method of claim 12, wherein after formation said membrane is neutralized to a pH of about 7.3 to about 7.5.

15. The method of claim 1, wherein said irradiation is selected from the group consisting of electron beam, UV light and gamma irradiation.

16. The method of claim 15, wherein said irradiation is applied in process or post process.

17. The method of claim 15, wherein said irradiation is from about 1 to about 100 kGy or from about 10 to about 50 kGy.

18. The method of claim 1, wherein said porosity is from about 1% to about 90%.

19. The method of claim 1, wherein said porosity is from about 50% to about 80%.

20. The method of claim 1, the method further comprising coating the cross-linked, edible, porous hollow fiber or sheet membrane with a coating to enhance cell adhesion.

21. The method of claim 20, wherein said coating is selected from one or more of fibronectin, fibrinogen, laminin, collagen, gelatin or short peptide sequences isolated from those proteins.

22. The method of claim 21 wherein said short peptide sequences are selected from one of more of the group consisting of RGD, YIGSR, IKVAV, DGEA, PHRSN and PRARI.

23. The method of claim 1, the method further comprising modifying the outer surface of the cross-linked, edible, porous hollow fiber to enhance cell adhesion.

24. The method of claim 1, the method further comprising coating the cross-linked, edible, porous hollow fiber or sheet membrane with a plasticizer.

25. The method of claim 23, wherein said surface modification is selected from one or more of plasma, corona, abrasion, etching, ablation, or sputter coating.

26. The method of claim 1, wherein said proteins are powdered or finely milled prior to their dissolution in the solvent.

27. The method of claim 1, wherein said proteins are at least 70%, 80%, 90%, 95%, 98%, 99%, 99.9% pure.

28. The method of claim 1, wherein said polysaccharides are at least 70%, 80%, 90%, 95%, 98%, 99%, 99.9% pure.

29. The method of claim 1, wherein the ratio of protein to polysaccharide in said mixture is from approximately 10:1 to approximately 1:10 or approximately 1:99 to approximately 99:1.

30. The method of claim 1, wherein the ratio of protein to polysaccharide in said mixture is approximately 4:1 to approximately 1:4.

31. The method of claim 1, wherein the ratio of protein to polysaccharide in said mixture is approximately 1:1 or approximately 7:1.

32. The method of claim 1, wherein the formation bath comprises one or more of calcium, zinc, magnesium, iron and potassium, in combination with one or more of i) water, acetic acid, citric acid, lactic acid, phosphoric acid, malic acid, tartaric acid, or one or more of ii) sodium hydroxide and potassium hydroxide.

33. A hollow fiber or sheet membrane made by the method of claim 1.

34. A method for manufacturing cross-linked, edible, porous hollow fibers or sheet membranes, comprising:
a) providing: i) one or more edible proteins, ii) one or more edible polysaccharides, iii) one or more solvents and iv) a formation bath, wherein the formation bath comprises one or more of calcium, zinc, magnesium, iron and potassium, in combination with one or more of 1) water, acetic acid, citric acid, lactic acid, phosphoric acid, malic acid, tartaric acid, or one or more of 2) sodium hydroxide and potassium hydroxide;
b) co-mixing the one or more edible proteins and one or more edible polysaccharides in the one or more solvents to form a mixture;
c) extruding the mixture into the formation bath to form an extruded hollow fibers or casting the mixture to form a sheet membrane; and
d) exposing the extruded hollow fiber or sheet membrane to an energy source selected from one or more of heat and irradiation sufficient to at least partially crosslink the one or more proteins to form cross-linked, edible, porous hollow fibers.

35. A hollow fiber or sheet membrane made by the method of claim 34.

36. The hollow fiber or sheet membrane of claim 33, wherein one or more proteins, one or more polysaccharides, one or more solvents, plasticizer and/or one or more constituents of the formation bath is generally recognized as safe (GRAS) by the U.S. Food and Drug Administration (FDA)

37. The hollow fiber or sheet membrane of claim 33, wherein the resulting sheet membrane or hollow fiber undergoes a 10-50% glycerol in water exchange for drying without pore collapse.

38. The hollow fiber or sheet membrane of claim 35, wherein one or more proteins, one or more polysaccharides, one or more solvents, plasticizer and/or one or more constituents of the formation bath is generally recognized as safe (GRAS) by the U.S. Food and Drug Administration (FDA).

39. The hollow fiber or sheet membrane of claim 35, wherein the resulting sheet membrane or hollow fiber undergoes a 10-50% glycerol in water exchange for drying without pore collapse.
